(19) United States
(12) Patent Application Publication
(10) Pub. No.: US 2019/0222323 A1
(43) Pub. Date: Jul. 18, 2019

(54) APPARATUS AND METHOD FOR ATOMIC FORCIPES BODY MACHINE INTERFACE

(71) Applicant: Peter Robert Butzloff, Saint David, ME (US)

(72) Inventor: Peter Robert Butzloff, Saint David, ME (US)

(21) Appl. No.: 15/826,467

(22) Filed: Nov. 29, 2017

(51) Int. Cl. H04B 10/90; G06F 3/01; B82B 1/00; A61L 27/08; A61B 5/0245; A61N 1/05; A61N 1/04; A61K 51/02; A61L 27/02

(52) U.S. Cl. CPC H04B 10/90; G06F 3/011; B82B 1/002; A61L 27/08; A61B 5/0245; B82Y 5/00; A61N 1/05; A61N 1/0468; A61K 51/02; A61L 27/025; A61N 1/0587

(57) ABSTRACT

A metamaterial structure, forming an atomic forcipes, including a topological conductor, a topological insulator abutting the topological conductor, and a gallery between the topological conductor and the topological insulator. The topological conductor has deuterons as chemical adducts. The topological insulator expresses a net negative surface charge and has paramagnetic properties. The gallery has charged intercalated ions. The topological conductor includes deuterated ferromagnetic graphene sheets. The topological insulator can include a clay sheet disposed between the graphene sheets. The atomic forcipes includes a nuclear magnetic isotope disposed in the gallery and formed as an adduct to the clay sheet. The atomic forcipes includes a transceiver, a transmitter, a receiver, a sensor, or an actuator. Included is a body-machine interface where atomic forcipes is disposed in or on a biological structure. The atomic forcipes transceives acoustic signal or electromagnetic signal, corresponding an ionic signal or an electrical signal in the biological structure.

### CROSS-REFERENCE TO RELATED APPLICATIONS

This application is related to U.S. application entitled “ATOMIC FORCIPES AND NUCLEAR MAGNETIC ISOTOPE SEPARATION METHOD AND APPARATUS” Attorney Docket B054-8010, filed concurrently, on even date herewith, which is co-pending with the present application, and which hereby is incorporated by reference in its entirety.

### FIELD OF INVENTION

The present invention relates to wireless interface devices, and more particularly, to a wireless nanodevice interface configured to communicate with biological structures.

### BACKGROUND OF THE INVENTION

Information transfer and processing of thought in biological systems among glia and neurons proceeds by a slow buildup or a rapid release of transferred ions, accompanied by electrical voltage spikes on the order of milliseconds. Both the ionic content as well as the voltage pattern contains useful information. Artificial computational devices have focused on the transfer of electrons, the spin state of electrons or atomic nuclei, and electromagnetic radiation to perform both information transfer and processing of information. It is desired at some point, to form improved interfaces between artificial computational machines and the natural biological equivalents to allow rapid mutual information transfer and processing to take place between traditionally biological and traditionally electromechanical systems.

Recently, quantum dots have been used to study neuronal receptors; however, commercially available graphene quantum dots are in general 25-35 nm in diameter and require a size reduction process or electrophoretic sorting to overcome size-related hindrance that may otherwise prevent their entry into the neural synaptic cleft, where this cleft has a gap that is typically about 20 to 30 nm wide.

Impurity doping can be used to dramatically alter the physical properties of nanostructures in metamaterials to enable novel technological applications. In general, spin-coupling and magnetic coupling exchange effects can be enhanced by geometric confinement in nanometer-scale structures. Nuclear magnetic resonance was first achieved in atoms by (i) keeping the external magnetic field strength constant and varying the frequency of radiation to seek a narrow region of absorption, where such frequency is called an absorption line; and by (ii) keeping the radio frequency constant and slowly varying the field strength until the splitting of spin states corresponds to the energy of radio waves associated with an absorption line. As early as 1946, the Purcell Effect was discovered, wherein a collection of small closely-spaced metallic antennas of about one-micron size was theoretically proposed to reduce the size of both receiving and transmitting antennas by creating a state of spontaneous resonance with nuclear magnetic materials placed in close proximity.

In modern times, the resonance states of atoms can be probed using an intense radio frequency pulse. This is used to excite all nuclei simultaneously so that their individual absorptions can be determined at one time, using the Fourier transform method. The latter method is of significant advantage to discover the instant state of spin orientation of the atomic nuclei as these interact with the surrounding electrons, each of which also generates a small local magnetic field. The local magnetic fields of electrons can either oppose or augment the external magnetic field. If the field created by an electron opposes the external field, the transmitted energy is attenuated, such that nearby nuclei obtain an effective field smaller than the external applied field, resulting in a shielding effect. If the field created by the electron augments the external field, nuclei produce an effective field which is larger than the external field, where this amplification of signal is called de-shielding. Both shielding and de-shielding together contribute to line broadening because the magnitude and direction of the angular moments generated by random electron spins contribute to the randomization of the atomic precession of nuclei. These effects become greater when the density of the randomly spin-oriented charge carriers becomes greater.

Nuclear magnetic isotopes (NMI) absorb and re-emit energy at certain combinations of radio frequency and magnetic field strength called the resonance frequency. When the applied radio frequency is directional, the re-emission of energy at that frequency is omnidirectional. This random energy scattering effect into all possible directions is the source of fluorescence and radio frequency absorption or line-width in the path of the externally applied directional energy. NMI can have an odd number of neutrons in their nucleus, and therefore have a net spin associated with that type of atom. When these NMI become charged, the ionic NMI generates a magnetic dipole along their spin axis. The magnitude of this dipole is called the nuclear magnetic moment. The nuclear magnetic moment has a greater value when the spin quantum number associated with the structure obtained by a particular number of protons and neutrons is a greater value. The magnetic dipoles of NMI are diamagnetic.

The electronic structure exerts a stronger magnetic field than the nuclear influence; therefore, the magnetic behavior of an element must consider the electrons as the primary contribution to the magnetic property of any ion. The 2.2 percent natural abundance of the 57-Fe isotope makes it very rare in the blood or the brain. Even at low radio frequencies of 3.23 MHz, there is good separation of 57-Fe from hydrogen (reference frequency at 1 MHz). Another atomic isotope intercalant for NMI neural probes NOT having ferromagnetic or superparamagnetic properties, but having useful high diamagnetic quadrupole moment, include cobalt as 59-Co, the two non-radioactive odd-atomic mass isotopes of Copper being 63-Cu and 65-Cu, and 55-Mn. A list of isotopic ratios in nature can be found at https://www.webelements.com/iron/isotopes.html. The environment of any of these isotopes can be accessible to pulsed electron spin resonance (ESR) analysis. The cooperative nuclear spin transitions are most sensitive to detection by modulation of the electron spin echo signal. A review of electron spin echo modulation (ESEEM) analysis is available at http://www.epr.ethz.ch/about-epr-research-group/what-are-spin-echoes/spin-echoes/eseem.html. Proton spin relaxation rate in proton nuclear magnetic resonance imaging can depend on the dipolar interactions between a NMI and a proximate proton having a spin, as well as on the distance between each of these to provide information about their distribution, using the technique of electron-nuclear double resonance spectroscopy (ENDOR). ENDOR types are explained at https://chem.libretexts.org/Core/Physical_and_Theoretical_Chemistry/Spectroscopy/Magnetic_Resonance_Spectroscopies/Electron_Paramagnetic_Resonance/ENDOR%3A_Theory.

Two effects originate in graphene of one atomic layer thickness that has achieved a significant commercial interest in nuclear magnetic resonance. The externally applied field causes an induction current. The inductor coil in old-time electronic circuits had a three-dimensional character; similar induction into graphene is confined to the delocalized pi-electrons above and below the carbon sheet of the graphene, where the circulating electrons achieve a ring current of maximal voltage near the edges of the sheet. The circulating graphene ring current creates a local magnetic field that can act as both a shield and a de-shield for any ions that are proximate to that sheet. This effect of graphene occurs on both physi-sorbtion and chemi-sorption of hydrogen, and is maximized when up to about 50% of delocalized sp-2 carbon-carbon bonds are reacted with hydrogen to form sp-3 bonds; the magnetization that arises because of local out of plane deformation of the graphene sheet and a directional density of electron states is called diamagnetism. More specifically, the planar or two-dimensional character of graphene makes it a special case of diamagnetism called anisotropic diamagnetism, because the magnetic field is highly directional, being normal to the sheet of carbon atoms. The basic types of magnetic behavior in ions of each element may be diamagnetic, ferromagnetic, anti-ferromagnetic, and paramagnetic. A list of the type of magnetic behavior for each element is available at http://www.periodictable.com/Properties/_A/MagneticType.html. Paramagnetism is magnetic attraction associated with an applied magnetic field. Oxygen and nitrogen are examples of atoms obtaining paramagnetic properties when they lose one of their unpaired electrons.

Clay materials are substantially composed of oxygen, with expression of oxygen atoms provided with unpaired electrons at the external surfaces. A clay sheet is not magnetic; however, clay contains oxygen atoms with two unpaired electrons at its surface. Irradiation by microwaves or by UV light can promote one electron per oxygen atom to a sufficient energy that allows complete separation from its parent atom, leaving an oxygen free radical having one lone electron in its outer orbital. This confers a paramagnetic property to the oxygen atom. The outside surface of clay has a net negative charge because of fixed charge imbalances arising from positively charged impurities inside the clay sheet structure. Clay sheets therefore have the interesting property of being able to transform into negatively surface-charged paramagnetic particles.

Graphene is a two-dimensional quantum spin Hall conductor, which may turn into a topological conductor at low temperatures when a magnetic field is applied perpendicular to the plane of the field because of Dirac electrons present in the graphene sheet. Without a sufficiently strong magnetic field applied normal to the graphene sheet, and at room temperature, graphene is a normal conductor. When graphene becomes reacted with hydrogen or deuterium, it becomes a ferromagnetic material. When the applied magnetic field varies in magnitude and reverses in direction, the graphene sheet acts as a two-dimensional inductive element in an electric circuit. This type of charge transport is different than in traditional bulk metallic conductors, where the transport of charge expresses no preferred quantum spin state orientation during the transport process. The benefit of such spin selectivity is to prevent electron backscattering from atomic scale disruptions in the motion of the electrons arising from imperfections such as folds or cracks that would ordinarily disrupt electron transport through such a thin layer of the material. These conditions usually only arise in a single graphene sheet at very low temperatures and at very high magnetic fields.

A topological insulator is a solid substrate able to allow constrained charge transport only at its surface, wherein the spin-orientation of the transported electrons and holes are in alignment. Topological insulators have conducting surface states protected by time-reversal symmetry, wherein electron spins and holes are locked at right angles to their momentum or direction of travel. The quantum Hall effect normally happens only at very low temperatures, close to absolute zero. Inside the material, electrons move in small circles called Lamour orbitals, but around the edges of a planar topological insulator, electrons can only move in one direction, like the case of the topological conductor.

Efforts to curb the random precession of multiple atomic nuclei have met limited or no success, and this extends to efforts to use nuclear magnetic isotopes as neural probes. As a result, neural probe technology has been significantly limited to omnidirectional energy output such as fluorescence from graphene or from gyrating dye polymers containing light emitting chromophores, or combinations of graphene with voltage-gated dyes to emit photons of a desired wavelength; the energies produced scatter into all different directions. The use of wires made from bulk metals to conduct electricity requires invasive voltage measurements of neurons that are associated with conductive probes for current and voltage. The obtained measurements of voltage and current contain no useful spin-current information. Indeed, spin-currents are not even envisioned or conceived to have any applicability whatsoever to the development of future brain machine interfaces now. The use of graphene alone as a neural probe substrate can provide anisotropic emission of infrared radiation, but these radiations are thermal in nature and heating is usually damaging or undesirable when measuring or interfacing with biological tissues. While tuning effects can be achieved by selecting the incident radiation and the particle size, graphene alone is unable to report on the full extent of information required to construct a brain machine interface or BMI.

Economical methods of restricting or mitigating random nuclear magnetic precession of NMI by collectively orienting their electronic spins require development of charge scattering control and longer mean free path lengths before charge recombination to enable remote wireless sensing in graphene based metamaterial sensing devices. This is especially important to enable neural probe technologies to monitor or influence the electric charge transport and ionic species being transported across the synaptic cleft or within the structures of neurons.

Vast shortcomings in medical and computational technology exist in creating an interface between prosthetic devices and biological neural structures, more generally known as the body-machine interface (BMI). While traditional probes of neural function do exist, their application is quite limited at present. One limitation is the need for wires or transparent fibers to send and receive optical or electromagnetic information to and from the specified areas of the brain, or the particular somatic nerves, that are to control such devices or to obtain feedback on the sensors in these devices. A non-invasive two-way communication probe has not yet been developed that might be able to wirelessly interact with individual biological neurons on scales as diminutive as the neural synapse, without destroying or causing severe scarring of sensitive neurological tissues. It has not yet been considered that any type of radioisotope would, or even could, factor into such considerations in the clarification and enablement of brain science.

### SUMMARY

Some present embodiments provide a metamaterial structure, forming an atomic forcipes, including a topological conductor, a topological insulator abutting the topological conductor, and a gallery between the topological conductor and the topological insulator. The topological conductor has a preselected concentration of deuterons as chemical adducts therein. The topological insulator expresses a net negative surface charge and having paramagnetic properties. The gallery has charged intercalated ions. In some embodiments, the topological conductor includes plural deuterated ferromagnetic graphene sheets of atomic layer thickness. In some embodiments, the topological insulator includes a clay sheet disposed between the graphene sheets. The atomic forcipes further includes a preselected nuclear magnetic isotope disposed in the gallery and formed as an adduct to the clay sheet. The atomic forcipes includes a transceiver, a transmitter, a receiver, a sensor, or an actuator.

In certain embodiments the atomic forcipes includes a wireless interface with a neuron. The atomic forcipes has a length of between about 5 nanometers to about 20 nanometers, and is configured to wirelessly receive electromagnetic or acoustic radiation from a neuron. The atomic forcipes has a length of between about 5 nanometers to about 20 nanometers, and is configured to wirelessly transceive with, transmit to, or receive from a neuron, electromagnetic radiation. In some embodiments, the atomic forcipes includes a transceiver having a length of between about 5 nanometers to about 10 microns, and configured to transceive information with a biological structure. In certain selected embodiments, the transceiver is a phase modulated transceiver. In other selected embodiments, the transceiver is an amplitude modulated transceiver.

Other present embodiments provide a body-machine interface (BMI), including a biological structure, and atomic forcipes disposed in or on the biological structure. The atomic forcipes includes plural deuterated ferromagnetic graphene sheets of atomic layer thickness, a clay sheet expressing a net negative surface charge, having paramagnetic properties, and disposed between and abutting the plural graphene sheets, and a gallery between each graphene sheet and the clay sheet, the gallery having preselected nuclear magnetic isotopes disposed in the galleries and formed as adducts to the clay sheet, wherein the atomic forcipes bidirectionally transceives information with the biological structure. In the BMI, the preselected nuclear magnetic isotope includes an intercalated cation. The intercalated cation includes Fe+2 and the nuclear magnetic isotope comprises 57-Fe, or Mn+2 and the nuclear magnetic isotope comprises 55-Mn, or Co+2 and the nuclear magnetic isotope comprises 59-Co, or Cu+2 and the nuclear magnetic isotope comprises 63-Cu and 65-Cu in a respective approximate atomic mass weight ratio of about 69:31. The atomic forcipes has a length of between about 11 nanometers to about 20 nanometers.

In other present embodiments, the atomic forcipes transceive at least one of an acoustic signal or an electromagnetic signal, corresponding to one of an ionic signal or an electrical signal at a portion of the biological structure. In selected embodiments, the biological structure further includes a neural structure having a sending axon terminal, a receiving axon terminal, and a synaptic cleft therebetween, a portion of the biological structure is the synaptic cleft, and the atomic forcipes are disposed proximate to the synaptic cleft and bidirectionally transceiving information traversing the synaptic cleft.

In embodiments, the atomic forcipes includes a nanomechanical magneto-electric (ME) antenna, in which the ME antenna receives oscillating electromagnetic (EM) waves, oscillating EM fields of the oscillating EM waves act to induce an oscillating electric field in the conductive graphene sheet of the ME antenna, the oscillating electric field induces an oscillating electric voltage across a substantially in-plane longitudinal aspect of the graphene sheet, the induced electric field oscillations react against a static electric field of abutting piezoelectric material, in which mutually attractive and mutually repulsive mechanical forces arise between the abutting parts of the atomic forcipes, wherein the mechanical forces oscillate in proportion to the induced fields to create phonons, and the ME antenna includes an RF activated ME antenna. In other embodiments, the BMI includes atomic forcipes, which includes a nanomechanical magneto-electric (ME) antenna where the atomic forcipes are acoustically-actuated, and where acoustic actuation further includes sonic waves provided to the atomic forcipes to stimulate magnetization oscillations in the graphene sheet of the atomic forcipes, where the sonic waves have a frequency of between about 20 Hz to about 2.0 GHz, and where the magnetization oscillations result in the radiation of electromagnetic waves from the ME antenna. In yet other embodiments, the BMI includes atomic forcipes, which includes a nanomechanical magneto-electric (ME) antenna, where the atomic forcipes are electromagnetically actuated, and where the electromagnetic actuation further includes electromagnetic waves provided to stimulate electromagnetic oscillations in the graphene sheet of the atomic forcipes, where the electromagnetic waves have a frequency of between about 2 Hz to about 500 THz, and where the electromagnetic oscillations result in the radiation of phonons.

In still other BMI embodiments, the atomic forcipes is a sensor proximate to the synaptic cleft, where the sensor detects a change in an electrolyte concentration in the synaptic cleft. In yet other BMI embodiments, the atomic forcipes further includes a transmitter configured to transmit a representation of a neural state to an external device, corresponding to the change in the electrolyte concentration in the synaptic cleft. In other BMI embodiments, the atomic forcipes further includes a receiver configured to receive a signal, which initiates the change in the electrolyte concentration in the synaptic cleft. In selected other embodiments, the atomic forcipes includes a sensor proximate to the synaptic cleft, wherein the sensor detects a change in an electrolyte concentration in the synaptic cleft responsive to a glia. In other selected embodiments, the atomic forcipes further includes a transmitter configured to transmit a signal corresponding to the change in the electrolyte concentration in the synaptic cleft propagated from the glia. In yet other selected embodiments, the atomic forcipes further includes a receiver configured to receive a signal which initiates the change in the electrolyte concentration in the synaptic cleft propagated to the glia.

Yet other present embodiments provide a body-machine interface (BMI), including a biological structure comprising a biological intermediate, and atomic forcipes coupled to, and disposed proximate to, the biological intermediate. The atomic forcipes include at least one graphene sheet having a preselected concentration of deuterons as chemical adducts therein, a piezoelectric clay sheet expressing a net negative surface charge and having paramagnetic properties, and abutted to the at least one graphene sheet, and a gallery between the at least one graphene sheet and the clay sheet, the gallery having an adduct of a preselected nuclear magnetic isotope formed therein. In embodiments, the BMI provides atomic forcipes configured to be a sensor to detect a physical characteristic of the biological intermediate and a transmitter to wirelessly report a representation of the physical characteristic, or a receiver to wirelessly receive a representation of an action to be taken relative to the biological intermediate and an actuator to motivate the action in the biological structure. In some receiver embodiments, the atomic forcipes is configured to be a receiver to wirelessly receive a representation of an action to be taken relative to the biological intermediate and an actuator to implement the action in the biological intermediate.

In some BMI embodiments, the biological structure includes a first biological portion and a second biological portion with the biological intermediate therebetween, and the atomic forcipes obtains a physical characteristic representation of the biological intermediate and transmits the physical characteristic representation to a controller external to the biological structure. In embodiments, the physical characteristic representation comprises one of joint configuration, and the joint is a knee joint, a hip joint, a shoulder joint, an ankle joint or a wrist joint. In certain selected embodiments of the BMI, the biological structure is a spinal joint, the first biological portion is a superior vertebra and the second biological portion is an inferior vertebra, relative to a longitudinal spinal axis, and the physical characteristic representation comprises one of joint configuration.

Other BMI embodiments provide the atomic forcipes to be configured as a receiver to wirelessly receive a representation of an action to be taken relative to the biological intermediate and an actuator to implement the action in the biological intermediate, wherein the biological structure comprises a heart, the biological intermediate comprises a selected portion of the myocardium, the physical characteristic is change in an electrical characteristic representative of at least a portion of a cardiac cycle sensed by the atomic forcipes, and the transmitter transmits the physical characteristic to an external controller. In the other BMI embodiments, the atomic forcipes receives from the external controller an electrical characteristic representative of an electrical impulse to be imposed upon the myocardium intermediate, and actuates to impose the electrical impulse upon the selected portion of myocardium intermediate. In still other BMI embodiments, the biological structure includes a skeletal muscle, where the physical characteristic is a change in an electrical characteristic, and the change in an electrical characteristic causes the skeletal muscle to contract, relax, or alternatingly both. The skeletal muscle contracts one of isotonically, isometrically, or isokinetically. In yet other BMI embodiments, the biological intermediate is a skin wound with sutures and the physical characteristic representation of the biological intermediate is wound integrity, wound tension, wound infection, wound dehiscence, or wound healing. In BMI embodiments, the biological structure is soft tissue or bone, where the physical characteristic is a change in an electrical characteristic, where the atomic forcipes receives from the external controller an electrical characteristic of an electrical waveform to be imposed on the wound and actuates to implement the electrical waveform in the wound to promote healing. In other embodiments of a BMI, the biological structure is soft tissue or bone, the biological intermediate is a cancer cell, the preselected nuclear magnetic isotope is a radioactive isotope, the atomic forcipes receives physical characteristic representation corresponding to release of the radioactive isotope from the gallery, and actuates to release the radioactive isotope proximate to the cancer cell to kill the cancer cell. In still other BMI embodiments, the biological structure is soft tissue or bone, the biological intermediate is a cancer cell, the preselected nuclear magnetic isotope is bound to an oncological pharmaceutical, the atomic forcipes receives physical characteristic representation corresponding to release of the oncological pharmaceutical and actuates to release the oncological pharmaceutical proximate to the cancer cell to kill the cancer cell.

These and other advantages of the present invention can be further understood and appreciated by those skilled in the art by reference to the following written specifications, claims and appended drawings.

### BRIEF DESCRIPTION OF THE DRAWINGS

Embodiments of the invention can now be described, by way of example, with reference to the accompanying drawings, in which:

FIG. 1 illustrates an edge-on view of atomic forcipes, showing cationic intercalants between a graphene sheet and smectite clay, in accordance with the teachings of the present invention;

FIG. 2 illustrates an edge-on view of atomic forcipes, showing free radical initiation on light irradiation of graphene and smectite clay sheets, in accordance with the teachings of the present invention;

FIG. 3 illustrates an edge-on view of atomic forcipes, showing ultrasonic irradiation applied during light irradiation of graphene and smectite clay sheets, in accordance with the teachings of the present invention;

FIG. 4 illustrates an edge-on view of atomic forcipes, showing adduct formation during ultrasonic and UV treatment between graphene and smectite clay sheets, in accordance with the teachings of the present invention;

FIG. 5 illustrates an edge-on view of atomic forcipes, showing adduct stability maintained between graphene and smectite clay sheets after removal of ultrasound, in accordance with the teachings of the present invention;

FIG. 6 illustrates an edge-on view of atomic forcipes, showing microwave assisted biased polarization and displacement of a graphene sheet between canted smectite sheets, in accordance with the teachings of the present invention;

FIG. 7 illustrates an edge-on view of atomic forcipes, showing microwave assisted alternative polarization and displacement of a graphene sheet between differently canted smectite sheets, in accordance with the teachings of the present invention;

FIG. 8 illustrates an edge-on view of atomic forcipes, showing cationic intercalants between a graphene nanometer sheet and phosphorus doped smectite clay, in accordance with the teachings of the present invention;

FIG. 9 illustrates an edge-on view of atomic forcipes, showing dissolved hydrogen in graphene, and two alternative cation types for intercalation between sheets of doped or natural smectite clay, in accordance with the teachings of the present invention;

FIG. 10 illustrates an edge-on view of atomic forcipes, showing organic cations after intercalation between sheets of doped or natural smectite clay sheets interposed between a graphene sheet, in accordance with the teachings of the present invention;

FIG. 11 illustrates an edge-on view of atomic forcipes UV-initiated unpaired electron free radical formation among intercalated organic cations, in accordance with the teachings of the present invention;

FIG. 12 illustrates an edge-on view of atomic forcipes RF polarization induced isotope pumping and nuclear magnetic isotope adduct formation among intercalated organic cations, in accordance with the teachings of the present invention;

FIG. 13 illustrates a process diagram for the synthesis of atomic forcipes for a body-machine interface, in accordance with the teachings of the present invention;

FIG. 14 shows a graphene sheet reacting with deuterium oxide under microwave irradiation or ultrasound to form ferromagnetic deuterated graphene in abutment with clay having been intercalated with divalent nuclear magnetic cations, in accordance with one embodiment of the present invention;

FIG. 15 illustrates deuterated ferromagnetic graphene sheet in abutment to a clay sheet having multiple exemplary 57-Fe nuclear magnetic ions, in accordance with the teachings of the present invention;

FIG. 16 illustrates an embodiment of a neural atomic forcipes probe including two deuterated ferromagnetic graphene sheets in abutment to clay sheet, in accordance with the teachings of the present invention;

FIG. 17 illustrates dielectric properties of intercalated calcium and sodium ions associated with clay sheet structures composing one part of atomic forcipes, in accordance with the teachings of the present invention;

FIG. 18 illustrates a body-machine interface process using 11 nm to 25 nm atomic forcipes having back gate reflectors operating within the synaptic cleft between two neurons, in accordance with the teachings of the present invention;

FIG. 19 illustrates a body-machine interface process using micron sized atomic forcipes for operation on or implanted within organ tissues in accordance with the teachings of the present invention;

FIG. 20 illustrates a remote time domain reflectometry (TDR) system for broadband pulse generation and Fourier transform processing, which may be configured for use with atomic forcipes in accordance with the teachings of the present invention;

FIG. 21 illustrates a neuron being irradiated by a remote TDR controller to obtain neuronal polarization information, in accordance with the teachings of the present invention;

FIG. 22 illustrates wireless active implantable neural devices (AIND) for muscle excitation and sensing functions, in accordance with the teachings of the present invention;

FIG. 23 illustrates atomic forcipes sensors and at least two characteristic active implantable medical devices (AIMD) for handling high RF power in cardiac tissues, in accordance with the teachings of the present invention;

FIG. 24 illustrates atomic forcipes sensors and characteristic active implantable neural devices (AIND) for brain machine interfaces (BMI) in brain tissues, in accordance with the teachings of the present invention; and

FIG. 25 illustrates atomic forcipes proximate to a biological structure and a remote controller used to transceive biological structure information, in accordance with the teachings of the present invention.

Some embodiments are described in detail with reference to the related drawings. Additional embodiments, features and/or advantages will become apparent from the ensuing description or may be learned by practicing the embodiment. In the figures, which are not drawn to scale, like numerals refer to like features throughout the description. The following description is not to be taken in a limiting sense, but is made merely for describing the general principles of the embodiments.

### DETAILED DESCRIPTION OF THE INVENTION

An atomic forcipes can be constituted of the juxtaposition of at least one paramagnetic clay sheet with at least one diamagnetic graphene sheet, together with a region of charged intercalated ions between these unlike sheets. The diamagnetic graphene sheet may be a topological conductor. The paramagnetic clay sheet may be a topological insulator.

Atomic forcipes can be configured to provide a wireless interface, which can be made with at least one substantially two-dimensional conductor being a graphene sheet of atomic layer thickness coupled to at least one substantially two-dimensional insulator such as a clay sheet expressing a net negative surface charge and having paramagnetic properties, and a critical concentration of chemisorbed deuterium on graphene of less than 50 percent coverage and greater than about 5 percent coverage or an average repeated atomic distribution of closer than about 6 unit cells in graphene, wherein deuterium provides at least one type of nuclear magnetic interaction to stabilize the topological phase by shifting the ferromagnetic phase boundary to enlarge quasi-topological regions in an incipient process. While protons can be reversibly dissolved into graphene, deuterons are not physically able to be dissolved into graphene. Deuterium atoms do not fit into the carbon lattice, being too bulky. They can, however, be bonded to carbon as adducts oriented normal to the carbon lattice plane, but not in a dissolved state. If too many carbons are bonded to hydrogen isotopes, this transforms many of the desirably delocalized electronic character of sp2 orbitals in graphene to sp3 bonded graphane. Graphane is not the same as graphene, and deuterated graphane is not conventional graphane. Atomic Forcipes as intended by the present invention preserves many of the delocalized bonds composing graphene.

A gallery (gap) exists between a graphene sheet and an adjacent clay sheet. Atomic forcipes may incorporate nuclear magnetic isotopes of any element into the gallery between an insulator material such as clay and a conductor such as graphene. This device operates with an intercalated ion, in particular, a nuclear magnetic isotope, in the presence of chemisorbed atoms of deuterium, free radicals at the surface of the clay, and an energy for activation consisting of acoustic, radio frequency, or light, to generate free radicals. Atomic forcipes doped with individual magnetic impurity ions can be induced to initiate ferromagnetic exchange coupling between delocalized charge carriers and individual impurity ions. The physical properties displayed by these materials may prove valuable for new wireless sensing and transmission technologies based on the manipulation of electronic and magnetic spin states in confined geometries.

The generation of the incipient state of topological spin-coupling uses a graphene sheet in proximate planar abutment to a deformed piezoelectric sheet of clay expressing a voltage and exerting a magnetic field normal to that voltage. This condition arises when the piezoelectric sheet is being deformed from one curvature to another, as by excitation energy provided by acoustic radiation or by a resonant electromagnetic radiation. Clay typically maintains an intrinsic internal static charge separation, however the piezoelectric voltage expressed as a difference of potential across the surface of the clay sheet ceases when the deformation ceases, which cascades to a loss of current expressed as a substantial charge carrier transport across the surface of the clay sheet.

An operating function of the atomic forcipes is to cause collective alignment of diamagnetic ions in the gallery between the clay and graphene under special conditions of applied frequency, where geometrically-confined nuclear magnetic isotopes at resonance are substantially spin-coupled with the charge carriers at the negative charged surface of the clay sheet insulator as well as with the quantum spin-coupled electrons in the graphene sheet. This coupling results in a directionally preferred isotopic chemical adduct reaction arising mostly at the clay surface with oxygen for cationic nuclear magnetic ions, as well as a less reactive directionally preferred adduct formation with nuclear magnetic isotopes at the graphene sheet. Externally applied irradiation creates a directional shield and de-shield of the magnetic field acting normal to the graphene sheet, as well as interactions with the electric field acting normal to the clay sheet surface. These electromagnetic interactions are each different in magnitude and in direction, but must follow a gradient with distance from each planar sheet to apply two angular moments that varies with proximity to each sheet. The combination of constrained motions of ions normal to the plane of the sheet with repetitive angular momentum torque acting to align random precession from each direction normal to the gallery region, serves to align the precession of nuclear magnetic isotopes in a dynamically cooperative spin coupling of these ions because of their planar alignment within the gallery region. In addition, the spin-coupling effects extend not only to the intercalated diamagnetic ions, but to the charge carriers of the graphene sheet in proximal abutment to the clay sheet. Therefore, currents induced into the graphene sheet by the applied irradiation become spin-coupled ring currents, where the spin coupling of the entire atomic forcipes acts together to provide substantially narrowed linewidths containing information about the atomic identity and chemical concentration of intercalants within the atomic forcipes structure. Atomic forcipes are useful, for example and without limitation, to encode or decode neural transmissions in the biological information transfer process. Atomic forcipes also are useful as a transceiver to wirelessly send and receive information relative to the atomic forcipes environment.

The combination of at least one clay sheet with at least one ferromagnetic deuterated graphene sheet, together with a region of charged intercalated ions between these unlike sheets, and an optional metallic backplane, especially for those particles of less than about 30 nanometers in diameter constitutes a neural atomic forcipes probe. The function of the metallic backplane is to provide a surface that is highly reflective to the incident irradiation; this consideration also provides a directional echo-response signal for time-domain reflectometry. When more than one receiver is present, the time-reflected pulse travel time is converted from duration to a time-of-flight signal travel distance that has the useful purpose of providing a directional triangulation of the atomic forcipes device within the living organism. The use of the THz frequency band is therefore not just useful for coherent transmission attenuation, but is especially advantageous for coherent pulse chirped time domain reflectometry (TDR). Frequency Domain Reflectometry also may be used, mutatis mutandi. Being far less energetic than those of x-rays, THz irradiation does not pose an ionization hazard for the biological tissue of the patient. Scattering of electromagnetic radiation in heterogeneous biological material is very complex, however it is many orders of magnitude less for the THz band than for the infrared or visible regions of the electromagnetic spectrum. Many biological molecules such as proteins, enzymes, and some neurotransmitters exhibit vibrational and rotational modes at THz frequencies that provide characteristic fingerprints to differentiate various types of biological tissues in this region of the spectrum that are very useful for medical applications, especially for those particles of greater than 1 micron in diameter constituting a body-machine interface atomic forcipes probe. In particular, the use of the Fourier transform analysis of the returned signal from a coherent directional chirped irradiation pulse leverages the large modal dispersion of a multiplicity of atomic forcipes in combination with their angular dispersion to create a chromatic dispersion spectrum essential for studying dynamical events such the effect of applied ultrasound, chemical dynamics in living cells, real time neural activity, and the microfluidics within the gallery regions of the atomic forcipes. The operating function of the atomic forcipes is to cause collective alignment of diamagnetic ions in the gallery between the clay and graphene under an applied frequency of irradiation, where nuclear magnetic isotopes at resonance are substantially spin-coupled with the quantum spin-coupled electrons. Currents induced into the graphene sheet(s) by the applied irradiation become spin-coupled ring currents, where the spin coupling of the entire atomic forcipes acts together to provide substantially narrowed linewidths of radio frequency absorption and emission associated with the intercalated sodium ions, potassium ions, and any proteins expressing transient conductive voltage by abutting the ions and conductive regions of the atomic forcipes structure. This process provides atomic forcipes with the wireless utility to encode by actuation of voltage and ions, or decode by remotely received electromagnetic emissions, the neural transmissions in the biological information transfer process.

NMI nuclei that have a hyperfine interaction with the electron spin system can be selectively monitored to result in an electron-nuclear double resonance (ENDOR) signal. This selectivity, for example, allows 57-Fe resonances in atomic forcipes to be distinguished from biogenic iron being substantially of 56-Fe isotope in hemoglobin or natural iron containing substances. Yet one more type of selectivity is acquired by the anisotropic planar orientation of the atomic forcipes. The position of the atomic forcipes provides a uniquely directional radio frequency signal that is normal to the plane of the atomic forcipes. This directionality allows the origin of the signal to be described in a given volume by setting multiple detectors across different angles. The sensitivity of the directional signal is enhanced for neural atomic forcipes of less than about 30 nanometers in diameter by the application of an electromagnetically reflective backplane to the dielectric clay component of the device. The presence of a signal at discrete angles separates the bulk response signal selectively from those of other atomic forcipes in the same volume having a different angle of repose. A magnetic pulse of sufficient intensity can serve to momentarily displace and substantially align the atomic forcipes with the direction of propagation of the magnetic field.

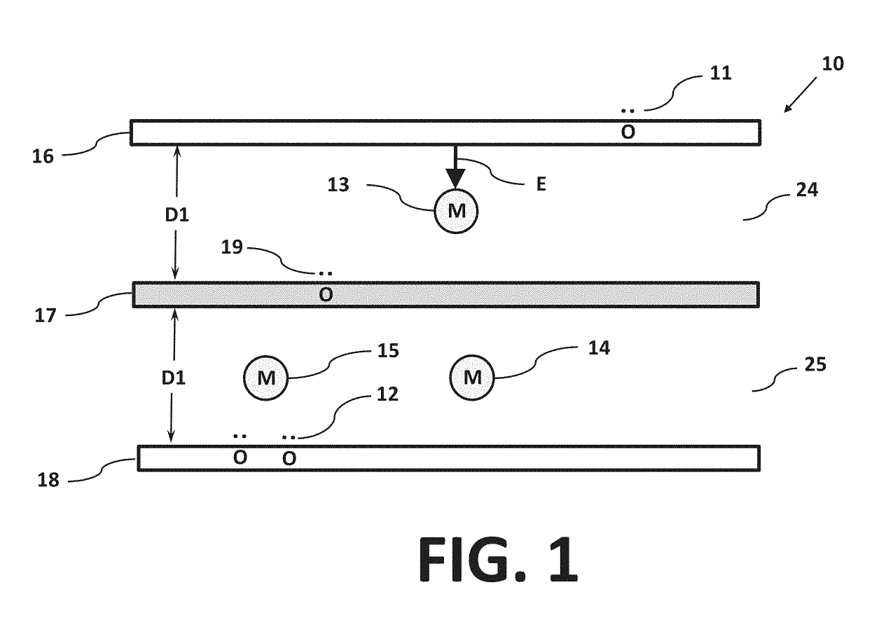

Referring now to the drawings wherein like elements are represented by like numerals throughout. In FIG. 1, a nanometer scale, trilayer atomic forcipes 10 is shown, in an edge-on view, to include a graphene sheet 17 of one carbon atom thickness, having occasional defects where a carbon atom is oxidized and replaced with an oxygen atom having a free electron base pair indicated by the two dots at 19. Graphene sheet 17 can be intercalated between clay sheets, including upper smectite clay sheet 16 having multiple exposed surface oxygen atoms with each such surface exposed oxygen atom having Lewis base electron pairs 11, and lower smectite clay sheet 18 also having multiple surface oxygen atoms with Lewis base electron pairs, represented in part by the two such oxygen atoms shown at 12. A non-nuclear-magnetic isotope atom 13 designating a cation M of positive charge, can be attracted by the inherent negative charges present in the monolithic clay sheet 16 by electric field E within the gallery 24, 25 between sheets designated by distance D1 of a few nanometers height. Herein, an exemplary ion can be magnesium, having two positive charges (Mg+2), of an isotope having atomic mass of 24 atomic mass units (AMU). Cation M 14 can be again a Mg+2 of 24 AMU, and cation M 15 can be a nuclear magnetic isotope Mg+2 of 25 AMU. Cation M of the indicated process using magnesium may be replaced in other processes by other chemical ionic species M having a chemical identity of calcium (Ca+2), etc., each having a range of natural isotopes both non-nuclear magnetic and nuclear magnetic, where the atomic forcipes can separate out the preferred nuclear magnetic isotopes when the magnetic isotope effect is applied among otherwise chemically identical isotopes in accordance with one embodiment. Atomic forcipes also can be bilayer, i.e., formed from one conductive graphene sheet, e.g., sheet 17, and one smectite clay sheet, e.g., sheet 16 or sheet 18. Multilayered atomic forcipes structures also are contemplated, with multiple conductive graphene sheets intercalated between multiple piezoelectric smectite clay sheets.

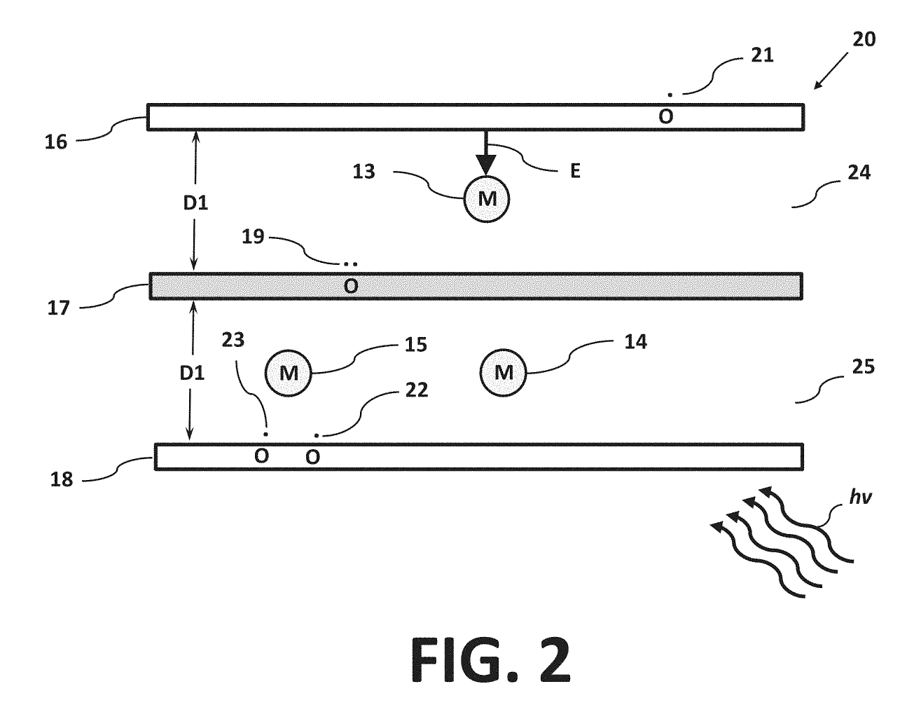

Referring now to FIG. 2 there is shown the nanometer scale atomic forcipes 10 of FIG. 1 after the introduction of high energy ultraviolet light indicated by symbol “hv” and expressed by the use of parallel wavy lines having arrows to indicate the direction of such illumination, where the passage of these rays has herein caused the removal of one of the paired electrons from some of the multiplicity of surface exposed oxygen atoms 21, 22, and 23 that are shown in an edge-on view of “free radical initiated” atomic forcipes 20, where the applied light irradiation transmits through and has interacted with some portions of graphene and smectite clay sheets. It is also understood that multiple atomic forcipes 20 typically can be used in a vapor or a solution process to separate out nuclear magnetic isotopes from a mixture containing large quantities of cations M, in accordance with one embodiment.

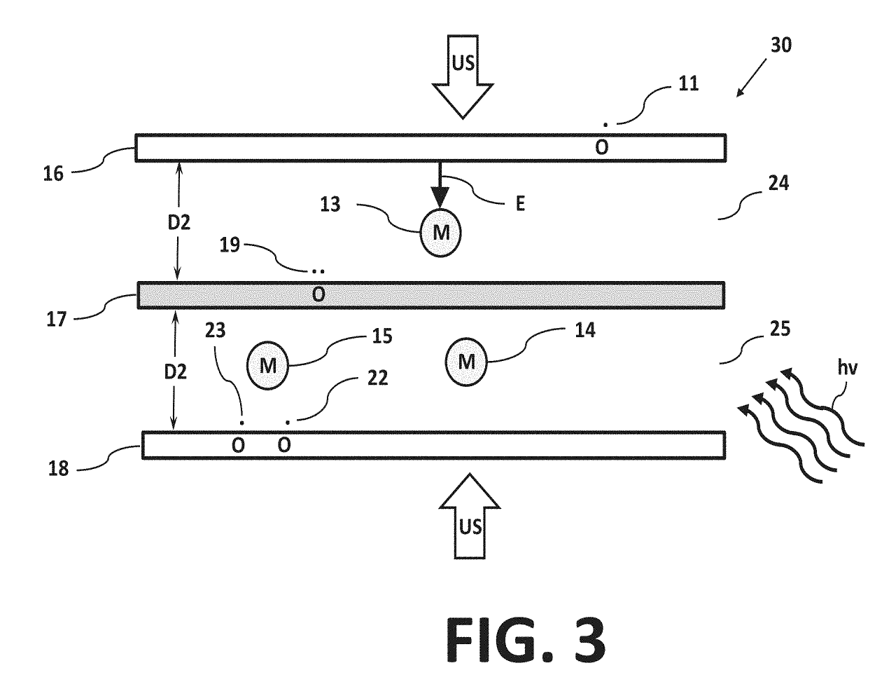

Referring now to FIG. 3 there is shown the nanometer-scale atomic forcipes 30, similar to atomic forcipes 20 of FIG. 2, after the introduction of ultrasonic energy indicated by the symbol US and a large arrow to show the direction of travel of these longitudinal phonon waves propagating parallel to this direction of energy transfer as a sequence of rarefactions and compressions applied using a desired frequency of about 20 KHz to about 2 GHz, but can sometimes be as low as sonic phonon frequencies ranging from about 20 Hz to about 20 KHz. This phonon frequency and magnitude can depend on process variables such as the chemical reactivity of the isotope ion mixture, the temperature of the process, the atomic radius and the number of atomic charges expressed by the processed cations M, and the density of an optional solvent carrier for fluidic transport of cations M. The solvent carrier can be, without limitation, any free-radical forming solvent such as water (H2O); an alcohol such as methanol, ethanol, or isopropanol; aliphatic hydrocarbons such as hexane, heptane, or octane; or aromatic hydrocarbons, such as benzene or toluene. The process also can depend on the number of alternating repeating sheets of graphene 17, and clay 16 which in sequential addition may comprise alternative larger atomic forcipes structures of otherwise functionally equivalent embodiments as the one herein shown in an edge-on view. Multiple several tens or hundreds of such stacked sheets results in considerable damping of phonon energy which may specify both frequency reduction and magnitude increase, where the primary effect and function of the sonic or ultrasonic irradiation US has momentarily and transiently reduced the representative gallery 24, 25 spacing during cyclic compression to an indicated distance of D2. This spacing reduction can result in a mutual proximate approach of indicated planar sheets 16, 17, 18 to the intercalant ions M applied during light irradiation of the indicated representative graphene and smectite clay sheets of atomic forcipes 30, such that the phonon induced repetitive oscillating pinching action in a direction normal to the plane of the sheets represents one type of actuation of these forcipes on an atomic scale, when applied in accordance with one embodiment.

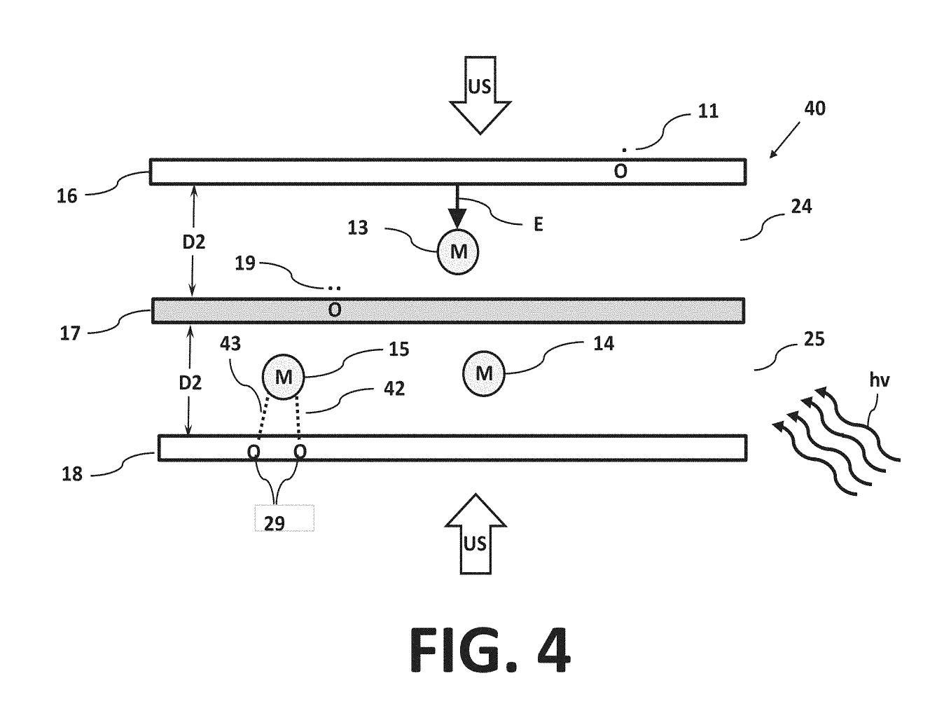

Referring now to FIG. 4, atomic forcipes 40 shows nuclear magnetic isotope M 15, reacted with a pair of oxygen atoms 29 in the substrate of the abutting clay lamina 18 by means of adducts 42, 43 having preferentially reacted under a highly confined but periodically altered geometry during ultrasonic treatment between graphene and smectite clay sheets, as the transient gap in galleries 24, 25 has reduced to the distance D2.

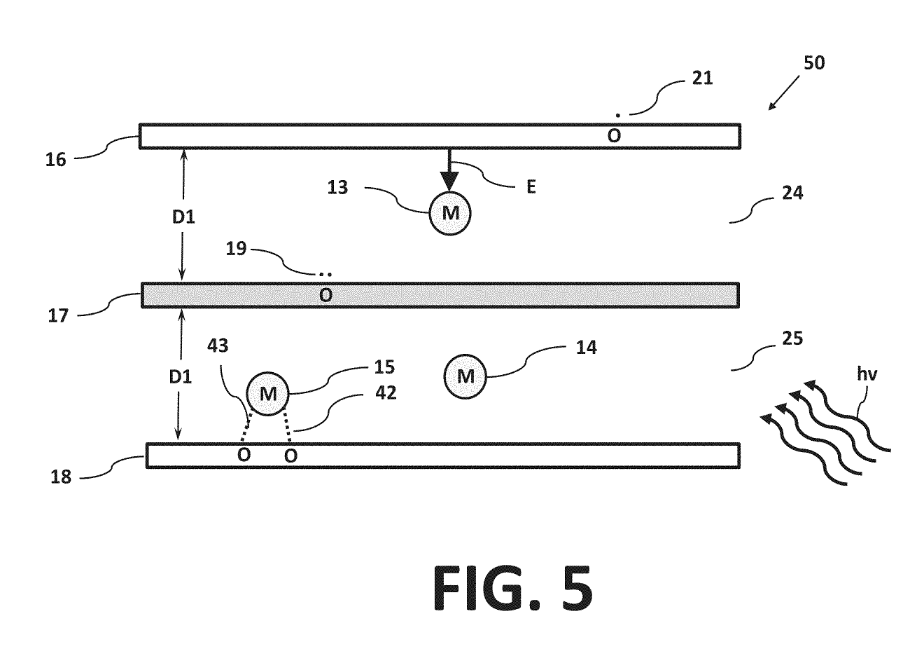

Referring now to FIG. 5 there is shown an edge-on view of atomic forcipes 50 demonstrating the adduct formed in FIG. 4, where the adduct bonds 42, 43 are maintained after ultrasonic actuation is removed and the gallery gap 24, 25 between graphene and smectite clay sheets has returned to distance D1.

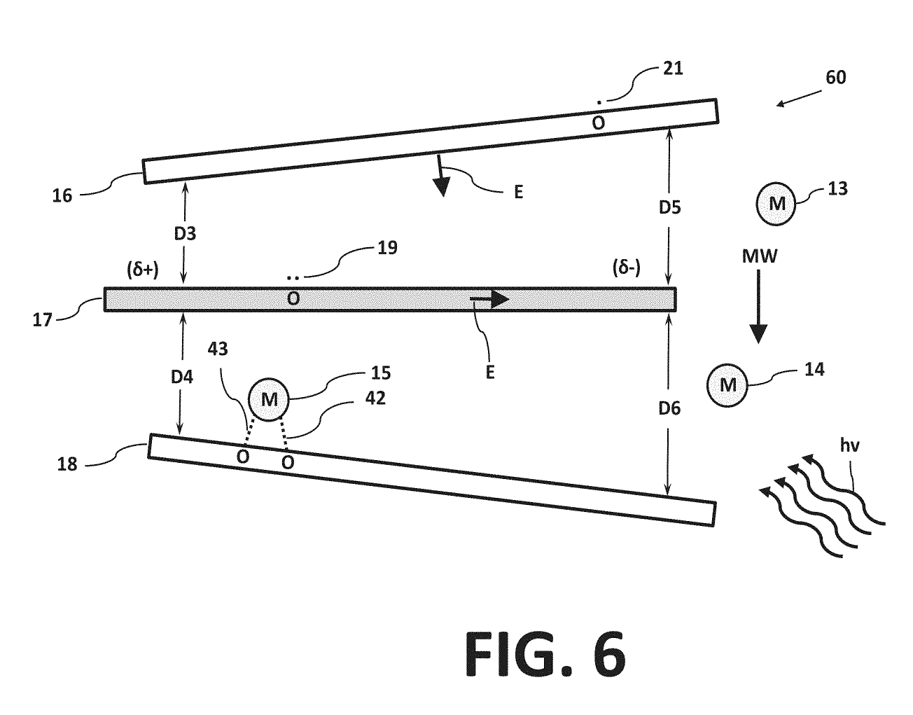

Referring now to FIG. 6 there is illustrated an edge-on view of atomic forcipes 60 showing microwave-assisted biased polarization and displacement of a graphene sheet 15, between canted smectite sheets 16 and 18, where the adduct bonds 42, 43 are maintained after microwave actuation. The induced charge difference across the graphene sheet indicated by the symbols (δ+) and (δ−) are the cause of a gallery gap reduction D3 and D4 in the presence of positive charged graphene regions, whereas the induced negative charge on the graphene sheet results in a gallery gap increase at D5 and D6. Cations 13 and 14 may now flow into the gallery indicated by D5 and D6. Transient electric field vector E is indicated by symbol E in the plane of the graphene sheet because of the radio frequency (RF) of applied microwaves indicated by symbol MW adjacent to the direction of an arrow to show the direction of RF wave propagation. The static or unchanging electric field vector E is indicated by symbol E normal to the plane of the clay sheets and is represented at clay sheet 16, where the entire atomic forcipes structure 60 operates in the manner of an electromechanical pump to provide such nanometer-scale displacements, in accordance with one embodiment.

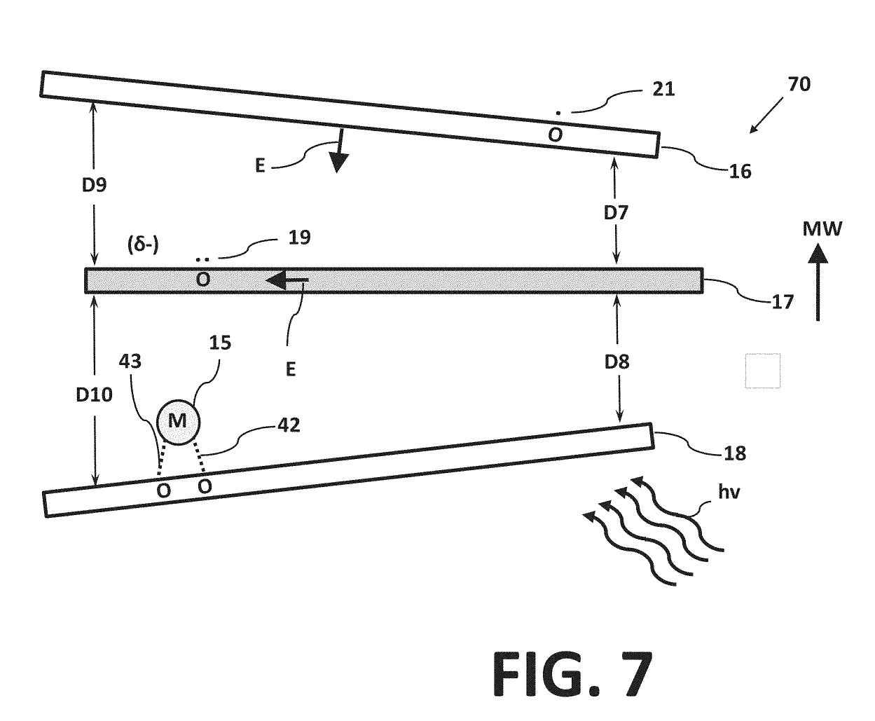

Referring now to FIG. 7, atomic forcipes 60 of FIG. 6 is shown in an edge-on view of atomic forcipes 70 demonstrating microwave-assisted alternative polarization and displacement of a graphene sheet between differently canted smectite sheets, where the direction of graphene sheet 17 polarization opposes that shown in FIG. 6, and where the transient or temporary electric field vector E within that graphene sheet 17 now opposes the direction shown in FIG. 6. A transient portion of microwave irradiative field propagation is shown by the arrow proximate to symbol MW to be pointing opposite to the one in FIG. 6. Non-bonding cations M are illustrated to be expelled from the proximate region of narrowed gaps indicated by the distances D7 and D8, where the entire atomic forcipes structure 70 operates in the manner of an electromechanical pump to provide such nanometer-scale displacements, in accordance with one embodiment.

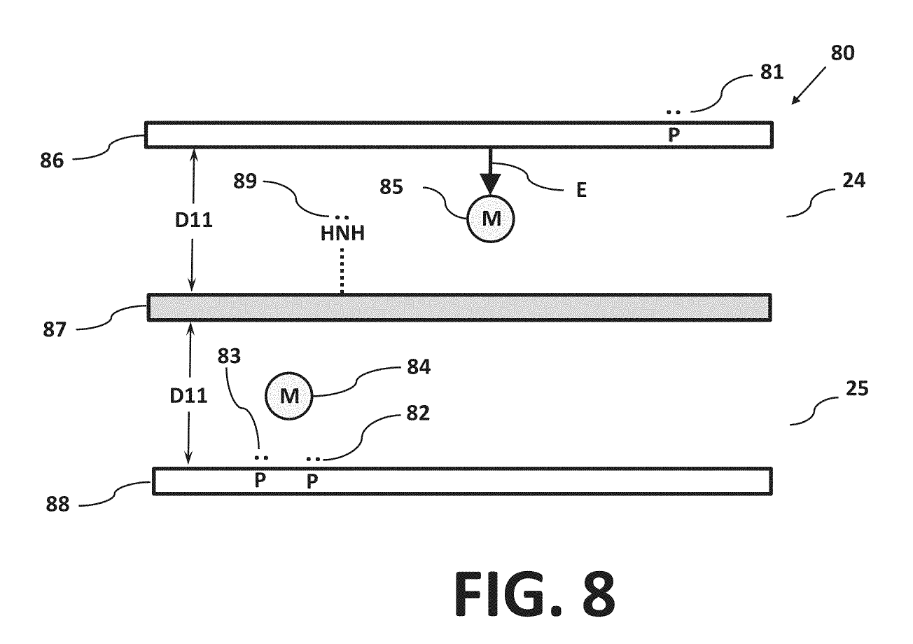

Referring now to FIG. 8 there is shown an edge-on view of atomic forcipes 80 depicting cationic intercalants M attracted to the static negative charges adjacent to clay sheets as expressed by the electric field vector E for cation M 85. Clay sheets 86 and 88 have been impacted by ion beams containing phosphorus to implant phosphorus surface impurity atoms among the oxygen atoms of the clay sheets, such that Lewis free electron pairs are expressed at the surface of these sheets by multiple phosphorus atoms 81, 82, 83. Implanted phosphorus is a nuclear magnetic isotope, 31-P, that is 100 percent abundant in nature; this implantation constitutes by way of illustration, one embodiment to introduce nuclear magnetic isotopes into the solid surface of atomic forcipes. Graphene sheet 87 has been functionalized by reacting it with ammonia to form an amine moiety provided with nitrogen having a free electron pair 89. The ability of multiple such phosphorus electron pairs at 81, 82, 83, and multiple such nitrogen electron pairs 89 now provide reactive sites for the possible future formation of an unpaired electron free radical at these atomic locations, in accordance with one embodiment.

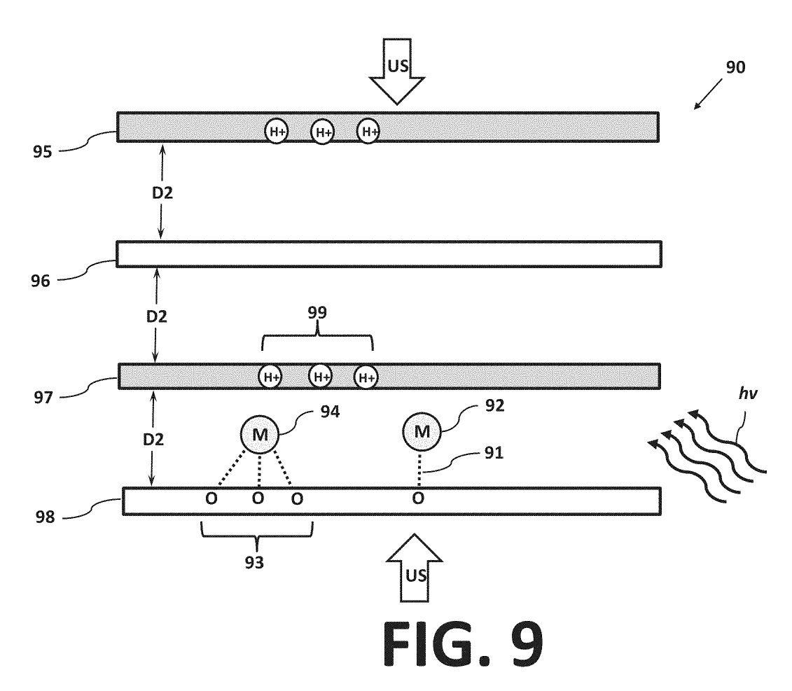

Referring now to FIG. 9 there is shown an atomic forcipes 90 exposed to ultrasound indicated by symbol US, exposed also to UV-light irradiation indicated by symbol hv, and is present in an environment having isotopes of hydrogen that result in the substantial dissolution of multiple positive charged hydrogen atoms 99 as depicted by the representative three dissolved hydrogen ions using symbols H+ in graphene sheet 97. Graphene sheet 95 has a similar population of dissolved hydrogen gas in this environment. Nuclear magnetic tritium is expressed by cation M at 92, and the adduct 91 is a hydrogen type bond with tritium to oxygen, where this tritium is substantially insoluble in graphene and cannot substantially dissolve into the indicated graphene sheets 95, 97. Nuclear magnetic deuterium likewise forms an adduct of a hydrogen type bond to oxygen, and do not substantially dissolve into graphene sheets 95, 97. Clay sheets 96 and 98 express negative charge arising within their internal structures and have oxygen surface atoms expressing free electron pairs, where three of these have formed the adduct 93 with a nuclear magnetic isotope atom selected from a periodic table group 3 cation M, 94, of valence expressing a positive 3 charge.

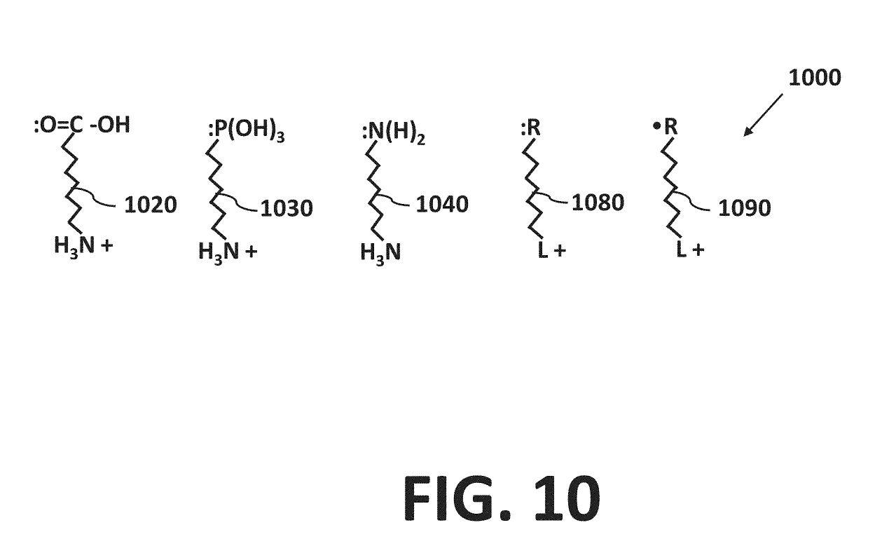

Referring now to FIG. 10 there is shown an edge-on view of organic alkyl cations having an ammonium functional group expressed by the atoms (H3N+). These positively charged molecular species at the end of an alkyl group representing a chain of carbon atoms are commonly termed alkyl-ammonium or alkonium ions, and they are used to replace natural sodium or calcium ions in common clay using an ion-exchange process. Each charge attracted replacement molecule is termed a ligand. With regard to proteins, a ligand can further be described as any molecule capable of binding to a protein with a high specificity and affinity. The ammonium functional group at the lower distal end of the molecule in 1020, 1030, 1040 is therefore designated L+ in 1080 and 1090 to represent a positive charged ligand that can later be electrostatically bound to a negative charged clay surface. A carboxylic acid functional group is shown at the upper distal end of the alkyl-ammonium molecule 1020, which has a pair of Lewis electrons on oxygen. Molecular deformation by ultrasound is only one type of compressive deformation of the organic intercalants; static or repetitive mechanical pressure as conferred by the pressure of bone and cartilage in a load bearing body joint will act to deform implanted atomic forcipes ion-exchanged intercalated polymeric adducts 1000, expel sodium ions M, and reduce the average gallery gap distance; moreover, high concentrations of atomic forcipes may be used intentionally to collectively amplify the signal response for pressure by altering electric percolation among a multiplicity of conductive graphene contact points, thereby altering the collective absorption and re-radiation of applied electromagnetic radiation.

A phosphate functional group is shown at the upper distal end of the alkyl-ammonium molecule 1030 which has a pair of Lewis electrons on phosphorus. An amine functional group is shown at the upper distal end of the alkyl-ammonium molecule 1040 which has a pair of Lewis electrons on nitrogen. The upper distal functional group on molecules 1020, 1030, 1040 are representative of a class of functional groups having a pair of Lewis electrons on at least one atom in that functional group, where this class of functional groups is herein more generally represented by the symbol R in proximity to a pair of dots to represent two paired electrons, as illustrated by the molecule 1080. The loss of one electron of this pair in 1080 results in the structure represented by the molecule of 1090 such that the single dot next to the R symbol now represents a lone electron state also termed a “radical”, and indicates a dangling or partially bonded state at R which is highly reactive. Molecules 1080 and 1090 can be used to represent multiple such ligands having been ion exchanged onto sheets of doped or natural smectite clay, or attracted to oxygen containing substituents at the site of defects on graphene, and can be illustrated to reside interposed into the gallery between planar abutting sheets, where the bulk of such ligands considerably increases the gallery distance between abutting planar sheets, thereby enhancing the exchange of mobile ions, solvent molecules, and gases into these enlarged galleries, in accordance with one embodiment.

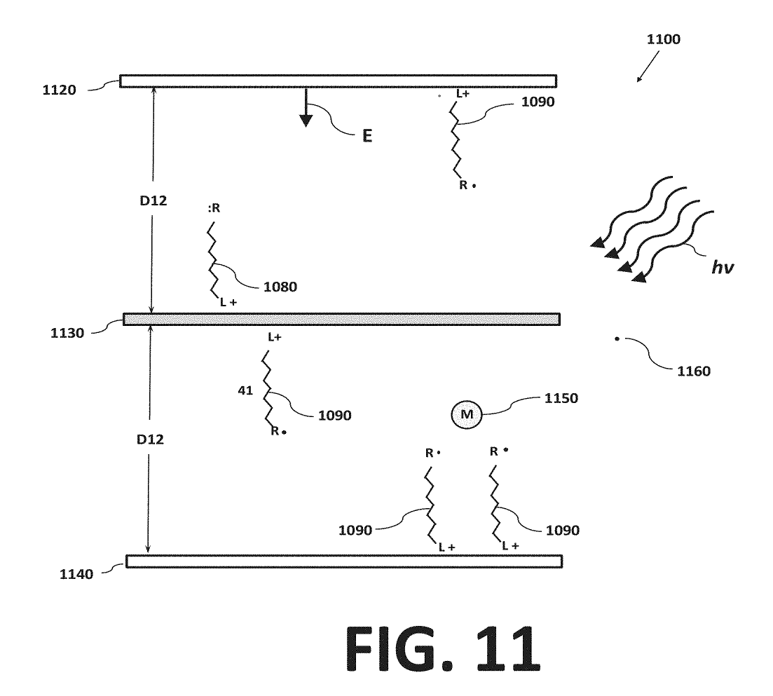

Referring now to FIG. 11 there is shown an edge-on view of atomic forcipes 1100 being irradiated by UV light expressed by a set of parallel wavy lines and the symbol hv. Static electric field vector E is shown directed normal to the plane of representative clay sheet 1120 where it is understood that such vectors can exist normal to either planar surface of each clay sheet including that of 1140. Graphene sheet 1130 is shown disposed between clay layers 1120, 1140. UV-initiated unpaired electron free radical formation can take place among intercalated organic ligands 1090. One hydrogen free radical 1160, a positive charged hydrogen atom is not dissolved into graphene, unlike the three dissolved hydrogen atoms shown in FIG. 9. Nuclear magnetic cation isotope M having positive 2 charges indicated as 1150 has easily and randomly migrated into the large gallery gap D12, in accordance with one embodiment.

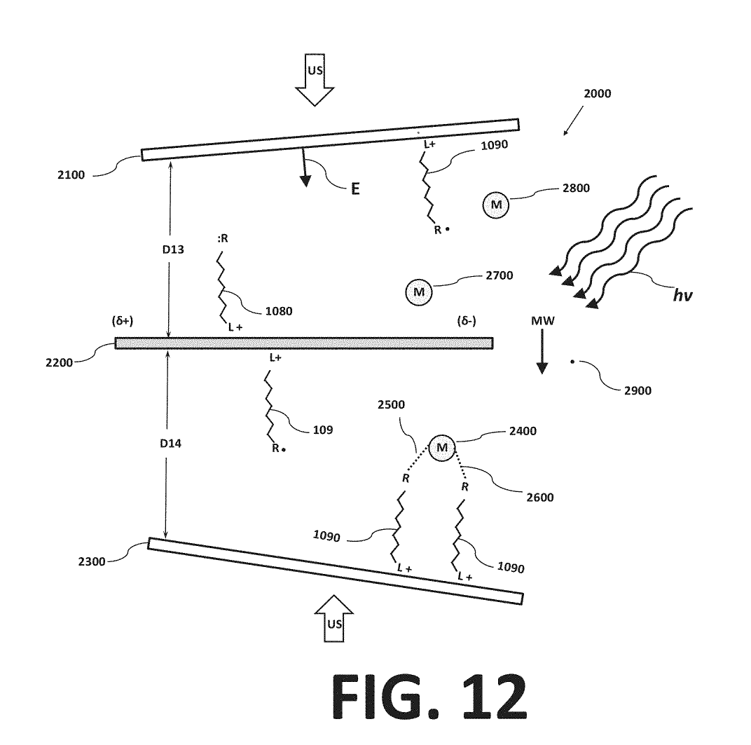

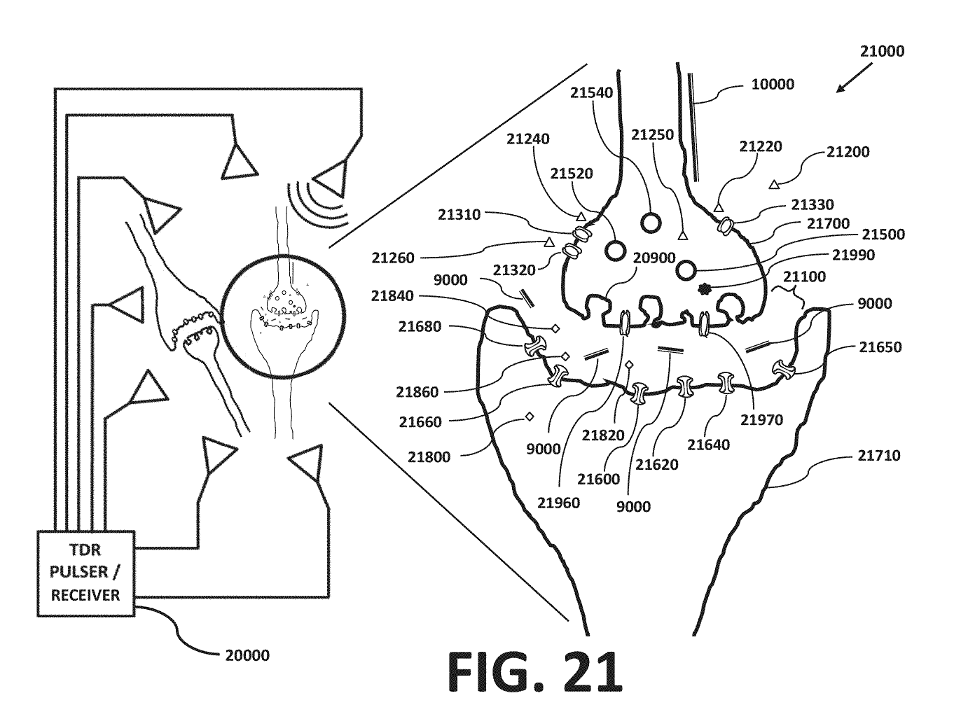

Referring now to FIG. 12 there is shown an edge-on view of atomic forcipes 2000 having a graphene sheet 2200 disposed between piezoelectric layers 2100 and 2300. In FIG. 12, forcipes 2000 can be undergoing RF polarization and inducing isotope M pumping of 2700, 2800, while under UV light irradiation free radicals are formed in aqueous solution 2900 and nuclear magnetic isotope adduct formation has been initiated at 2500, 2600 with intercalated organic cations 1090. Adduct formation is likely because of the tight packing of ligands 1090 within the constrained geometry of the gallery spaces D13 and D14 even if these express greater gaps than within galleries not exchanged with alkyl ligands. It is notable that ligands 1080, 1090 bend collectively in parallel at the charged sheet surface ligand site, indicating a reduced number of degrees of freedom compared with systems of randomly oriented reactive systems. A lessening of distance D13 and D14 will also occur under mechanical loading as shown by the environmental context which is illustrated by FIG. 21.

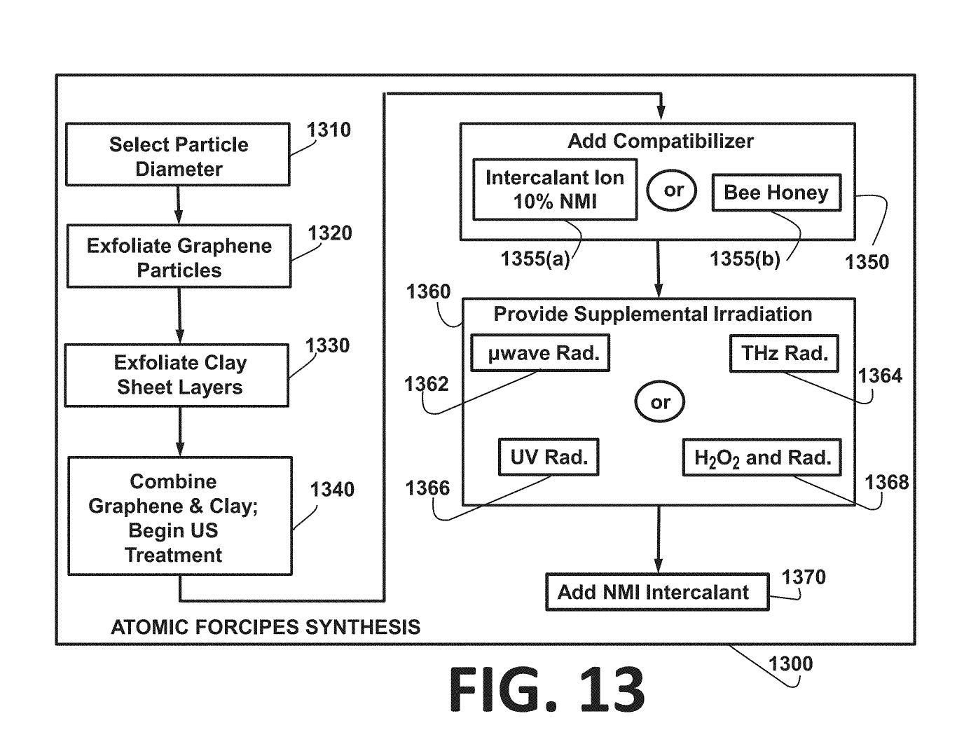

Referring to FIG. 13, an atomic forcipes synthesis method 1300 is described. Step 1310 of the synthesis is the particle diameter selection, where a typical thickness of any of these particles is about 1 nanometer and may be considered constant. The unlike nanoparticle materials can have well matched diameters. The application or purpose of the various embodiments determine a designated diameter to achieve the appropriate function.

For example, the cross-sectional area of atomic forcipes metamaterial involves the synthesis to begin using particles matched in diameter and tuned to interact with the light or radio frequency wavelengths of the irradiation desired for energy input. This can be achieved by starting with montmorillonite clay and natural flake graphite already sized to provide matched diameters. In yet another example, graphene particles obtained from exfoliation of graphite, as well as a combination of the above clay particles, can be deposited onto a monolithic (contiguous) graphene sheet of one atom thickness extending in size to as much as inches or more, wherein the clay dielectric particles are interposed between this monolithic graphene sheet and similarly sized discrete graphene particles having the scale of nanometers or microns. This type of geometry may be used as a skin sensor applied either above or embedded below the epidermis.

In another example, metamaterial particles are sized to maximize interaction with Terahertz irradiation, including infrared radiation. This may be achieved by the use of commonly available smectite clay mineral particles. Some sources of Laponite have a majority of (20-25 nm) diameter particles. Laponite is a synthetic smectic clay that forms a clear, thixotropic gel when dispersed in water. Alternatively, a clay having large diameter such as montmorillonite (100-150 nm) or Saponite (50-60 nm) or Hectorite (200-300 nm) is fractured using a standard ball milling process, and the particles are filtered or passed through a size exclusion sieve to provide diameters meeting the preferred diameter requirement. Alternatively, a bentonite clay is fractured using a high power ultrasonic irradiation for sufficient time to supply small diameter fragments that are sorted for a desired diameter. Bentonite, Saponite, and Hectorite are smectite clays of the montmorillonite group.

Graphene nanodots can be purchased to already meet a narrow diameter range (20-30 nm) for the particle diameter matching requirement useful for wireless neural probes. Graphene nanodots are commercially available and are typically produced using an electrolytic synthesis. One source of graphene nanodots can be ACS Material, LLC, Pasadena, Calif. USA.

Step S1320 in the synthesis 1300 is to exfoliate the appropriately sized graphene particles. The Woltornist interface trapping and exfoliation process is a method of obtaining sheet layer separated single-atomic thickness graphene for the production of atomic forcipes. For the Woltornist process, see Woltornist, et al., “Properties of Pristine Graphene Composites Arising from the Mechanism of Graphene-Stabilized Emulsion Formation,” Ind. Eng. Chem. Res. 2016, 55, 6777-6782, May 25, 2016, which document is incorporated by reference herein in its entirety. In the Woltornist process, graphene separation by mild ultrasound is the result of the strong affinity of the graphene sheets to the meniscus between immiscible highly polar solvent such as water, with an equal volume of low molecular weight non-polar solvent such as hexane or heptane. Either of these solvents, when used alone, are very poor dispersion matrices for graphene, as neither one can form stable suspensions with graphene nanoparticles without the presence of a liquid-liquid interface. The interface trapping and deposition technique is simple, inexpensive, scalable, utilizes practically any form of natural flake graphite or graphite nanodots with no prior treatment, and requires no post-treatments to obtain smectite sheet separation of graphitic layered raw materials. To have enough interfacial liquid surface area for individual sheets to remain separated in this process, one can use about 18 to 20 times the volume of mixed solvents to 1 volume of graphitic solid powder. However, interfacial liquid surface area can be expanded by the use of wide pans having a low liquid height to increase the effective surface of two immiscible solvents at the region of the liquid-liquid meniscus.

Step S1330 in this synthesis S1300 is the use of the Woltornist interface trapping and exfoliation process as a method of separating smectite clay sheet layers. Processing with ultrasound for about one hour obtains sheets of about 1 nanometer thickness at the polar and nonpolar solvent interface. Use of 1 part by volume of clay particles to 20 parts by volume of equal proportions of mixed polar and non-polar solvents can avoid clay sheet recombination. This process is not presently used to exfoliate clay; however, it is used here to provide process uniformity having no more than a few sheets of one type of particle available for interfacial reaction.

Step S1340 is to combine equal volumes of exfoliated graphene and exfoliated clay in polar and non-polar solvents, then immediately begin ultrasonic treatment of the combined mixture. The hybrid bi-layer sheets of conductive graphene particle and insulating electrostatic sheets of clay form a transient metaparticle association of atomic forcipes while ultrasound is being applied.

Step S1350, is to add compatibilizer for the particle surfaces by one of two alternative methods currently considered. In the first method 1355a, add an intercalant ion to the solution mixture containing about 10% nuclear magnetic isotope (NMI). This addition is made to the combined particles and solvents after about 10 minutes of ultrasonic treatment or when physical mixing of unlike particles appears homogeneous. The purpose of the NMI ionic intercalant is to form some hydrogen bonds at the surface of or between sheets of unlike particle materials before proceeding to step 6. This can stabilize the close association of unlike particle materials on cessation of the ultrasonic treatment. The selection of the NMI depends on the application of the embodiment. If the purpose of the atomic forcipes is to extract NMI silicon from silane, then a labile NMI ion is an appropriate compatibilizer to facilitate later ion exchange by displacement and replacement with the NMI of silicon. One NMI compatibilizer choice can be the addition of deuterium oxide (also known as heavy water) to the mixture of solvents and combined particles. Deuterated water forms a metastable hydrogen-bonded NMI capable of being removed by thermal de-bonding as heavy steam. If the purpose of the atomic forcipes is to create a solar reflectivity management material, then upper atmospheric metamaterial stability is desirable under ultraviolet irradiation, which may be achieved by the selection of natural magnesium isotope mixture to be added as a chloride salt to the combined particles until full reaction synthesis is completed. Natural magnesium contains about 10% of 31-magnesium NMI, which can become preferentially bonded as adducts to the free radicals at the sheet surfaces.

Alternatively, step S1355(b) is the addition of up to 1% by volume of natural bee honey to the mixture of solvents and unlike particles. This addition can be made to the combined particles and solvents after about 10 minutes of ultrasonic treatment or when physical mixing appears homogeneous. Natural bee honey can contain both polar and non-polar components that migrate to the appropriate solvent in this mixture. This migration by solubility enables a polar charge association of the clay particle surfaces and a non-polar Van-der-Waals-type of adsorption to the surface of the graphene particles. This option is available when residual honey contaminant is not an issue and does not detract from the desired function of an application. For example, honey at high concentrations can interfere with clay that has been ion-exchanged with ammonium diamines by masking of pendant Lewis paired nitrogen electrons required to form adducts with nuclear magnetic isotopes.

Optional step 1360 is a supplemental irradiation process to generate large numbers of free radicals. The purpose is to provide more opportunities for adduct formation than can be achieved at the selected ultrasound irradiation. This situation may arise if low amplitude ultrasound and short process times are desired. Adduct formation by free-radicals at the solid surface enhances the proximate stability of abutting surface sheets as bilayers of unlike particle materials to form them into the stabilized atomic forcipes metamaterial particle.

The following irradiations may be used alone or in any combination:

In step S1362, the solvent mixture with unlike particles is placed into an explosion proof microwave oven and subjected to microwaves at low power (about 100 watts) for less than 1 minute.

In step S1364, alternatively, the solvent mixture with unlike particles is placed into an explosion proof Terahertz resonant cavity chamber and subjected to Terahertz radio frequency waves, for example, a coherent infrared laser light, at high power (about 400 watts) for less than about 10 minutes.

In step S1366, alternatively, the mixture is placed into a chamber having half-submerged rotating discs to expose the particle layers to ultraviolet radiation above the surface of the immiscible solvents to activate the particle surfaces with free radicals. The transparency of two-layer sheets of graphene films is as high as 95%. Care is taken to make certain that the rotating disc penetrates the liquid-liquid interface or meniscus. The disc should be able to reach the layer where unlike particles have collected, to allow these to climb the disc surface. Selection of disc material favors quartz glass because of the transparency of quartz to UV-light and the ability of graphene to temporarily adhere to quartz. The use of glass discs is possible but blocks or limits UV and reduces the amount of UV light irradiating the particles between the discs. Such UV limiting discs can be spread apart a sufficient distance for UV light to reach all exposed disc surfaces above the solvent mixture. Particle films formed on rotating discs by graphene sheet climbing are consistently 4 or fewer sheets. The films formed at the bulk solvent interface, however, can be much thicker depending on the concentration of graphite. This discrepancy arises because of the sheet climbing phenomena being driven by reduced interfacial energy between polar solvent water absorbed on the hydrophilic glass walls being displaced once graphene occupies the glass interface. Once the glass is covered, then the driving force for climbing is diminished and no additional sheets may be drawn up.

In step S1368, alternatively, hydrogen peroxide can be added to the mixture as a free radical initiator in the presence of irradiative energy. Other commercially available free radical initiators may not be used because these do not readily decay into water and oxygen, and therefore may leave contaminating residues that interfere with the function of atomic forcipes and may otherwise be difficult to remove from the gallery of the metamaterial.

Optional synthesis step S1370 is to provide significant amounts of robustly surface-bonded NMI intercalant to the finally produced atomic forcipes. This is because the nuclear magnetic spin of high concentrations of NMI modifies the atomic forcipes out of plane flexural bending of the conductive sheet of graphene to comply with a specified frequency response as a sensor or a wireless transmitter. This synthesis is achieved for exemplary magnesium isotope mixture in one embodiment, with the understanding that another chemical identity atom type may be used in a similar synthesis. Natural magnesium chloride solution is introduced to the solvent mixture with atomic forcipes. Irradiation is allowed to proceed at low intensity and a sufficiently long irradiation time to allow diffusion of all isotopes of magnesium into the gallery, while permitting only the non-NMI ions to exit the gallery. The mechanism of non-NMI ion expulsion is by out of plane graphene sheet flexural flagellation in the presence of significant dynamic oscillations generated by irradiation. Such flexural energies are easily absorbed by the deformed adducts of NMI acting to absorb the energy of graphene sheet oscillation using the spin effect. The solution is then filtered to remove the particles, and the polar water solution is decanted and discarded, but replaced with fresh water and fresh dissolved magnesium chloride salt at about 1 to 10 percent concentration, which is added in aliquots. This step is repeated as often as required to displace non-NMI from the atomic forcipes gallery. Synthesis completion is determined by radio-frequency. This point is determined when the nuclear magnetic spin of high concentrations of NMI reduces the atomic forcipes out of plane flexural bending of the conductive sheet of graphene to form a substantially rigid graphene sheet that is able to absorb and re-radiate the applied radio waves. Because this synthesis step can be critical to applications with radio frequency emission functionality, a measure useful to determine reaction completion is explained in more detail as follows.

The acoustic and thermal properties of smectite or layered stacks of self-organized two-dimensional materials such as graphene or clay are extremely anisotropic. Phonons (sound energy) propagate rapidly in-plane where material modulus of the sheet is high, but much more slowly from one sheet to another across the gaps that separate sheets. The smectite structure allows anisotropic and interactive control of electromagnetic and ionic diffusion processes such as directional conductivity of energy that may be scattered between or among intra-facial ions and planar surfaces. The planar geometric anisotropy makes possible an elastodynamic wave propagation mode that leads to the precise control of wave trajectories in between the abutting sheets of metamaterials at fractional resonant wavelength nodes less than the parent excitation wavelength. Such emission can be achieved using a light laser pulse of about 800-NM or by irradiating the metamaterial with a radio-frequency pulse, then waiting for absorbance and re-emission.

An even more extreme control of an elastic wave field emitted in a thin sheet can be achieved by periodically pinning two abutting sheets at vertices of an array, such as by a honeycomb array of adducts. The result is a transformation of constructive and destructive interference at resonance of plasmon polaritons from three-fold symmetry to six-fold symmetry when the incident wave is shifted by only a few nanometers, or equivalently, when the gallery between pinned plates is adjusted by a few fractions of a nanometer. The latter case may arise when the local ionic concentration is suddenly changed in the region immediately outside the gallery, which gives rise to a change in the density of ions inhabiting the gallery by simple diffusion. This causes a frequency shift that is a useful property in nano-antennas and nanometer-scale sensors. Therefore, the addition of magnesium chloride (as little as 1 percent) may be sufficient to change the resonant radio wave emission characteristic, wherein this transition characterizes the NMI stabilized metamaterial particles of atomic forcipes. Stabilization is an easily detected quality factor that is very sensitive to ionic concentration changes, and constitutes the ability to significantly enable RF sensing and transmitting from atomic forcipes.

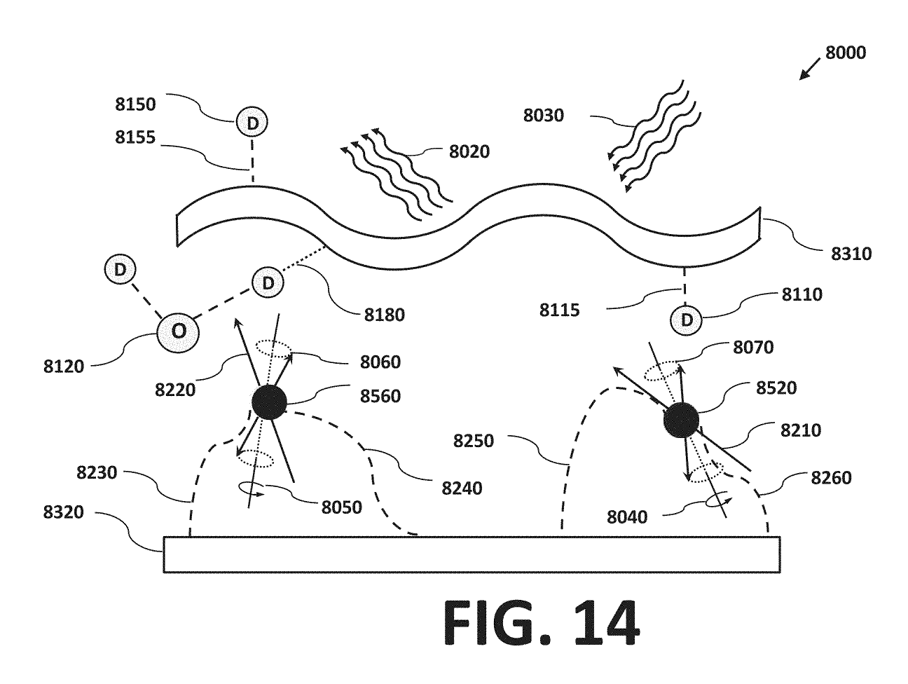

Referring now to FIG. 14, which can be representative of the deuteration of graphene 8000 in which deuterium oxide 8120 reacts with graphene 8310 in the presence of an energetic activation sufficient to form bonds 8155 and 8115 to multiple deuterium atoms represented by 8150, 8110 as a result of ultrasonic phonon actuation 8030 combined with optional microwave actuation and re-radiation 8020. Clay sheet 8320 has a characteristic negative static face charge with a countercharged population of nuclear magnetic ions such as 8520, 8560, for example, and without limitation, Mn+2, surface bonded as adducts to the exposed clay sheet surface oxygen atoms, introduced, for example, by the use of manganese dichloride (MnCl2) dissolved in deuterium oxide solvent. Fe+2 also can be dissolved in deuterium oxide solvent, using FeCl2. The chemically bonded adducts 8230, 8240, 8250, 8260 together with the planar geometric confinement of ferromagnetic manganese intercalants 8560, 8520 confer magnetoelectric material properties that couple with the ferromagnetic deuterated graphene sheet 8320. The multiplicity of nuclear magnetic ions 8520, 8560 have magnetic moments 8210, 8220 and electric moments 8040, 8050 affecting their precession 8060, 8070 coupled to their electromagnetic domain formation to assist in the attraction of competing charge carriers in graphene sheet 8310 that may otherwise interfere with the ballistic Dirac electrons required for resonance with externally applied irradiation 8030 for re-radiation at 8020.

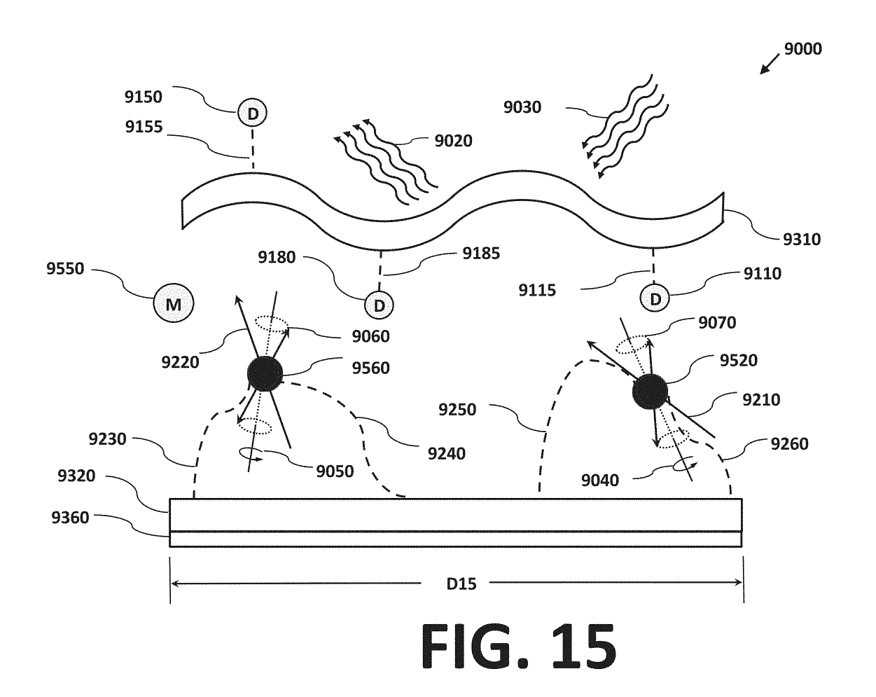

FIG. 15 shows deuterated ferromagnetic graphene sheet 9310 in abutment to a clay sheet 9320 having multiple exemplary 57-Fe (iron) nuclear magnetic ions 9520, 9560 bonded adducts, which each have magnetic moments 9210, 9220 and electric moments 9040, 9050 affecting their precession 9060, 9070 coupled to their electromagnetic domain formation to assist in the attraction of electron charge carriers. These electron charge carriers may otherwise interfere with the ballistic Dirac electrons in graphene sheet 9310 required for resonance with externally applied 9030 and re-radiated 9020 terahertz radio frequencies. Multiple sodium atoms 9550 marked M will be approaching and intercalating within the gallery between graphene 9310 and clay 9320 to interpose between the bonded deuterium and bonded nuclear magnetic isotopes 9560 and 9520 such that the loss component of externally applied Terahertz radio frequencies 9030 will be modulated at radio frequencies greater than 113 GHz for re-emission at 9020. In some embodiments, other neurologically active ions also transmitting current but not associated with neural firing potentials such as Ca+2, do not express the permittivity loss of sodium ions at these radio frequencies. Multiple chemical-adduct nuclear magnetic isotopes 9520, 9560 may be selected from any element of odd-numbered atomic mass having one more neutron than the count of protons to enable expression of coupling between the atomic electric field and the nuclear magnetic precession by their planar geometric confinement and stabilization through adduct bonds 9230, 9240, 9250, 9260. The clay sheet 9320 can have metal backplane 9360 to act as an electromagnetic reflector, for example, in synaptic region-placed neural atomic forcipes. This type of neural atomic forcipes can have a size of D15, which can be between about 5 nanometers to about 30 nanometers, and nominally about 11 nanometers in diameter, to fit, for example, into a neural synaptic cleft.

With the structure and function of atomic forcipes thus described, a wireless body machine interface (BMI) using atomic forcipes can be provided by embodiments herein. The BMI may be directional, and be amplitude modulated, phase modulated, or both. Although the atomic forcipes may be fitted proximately to a biological structure, they also can be fitted proximately to a neural structure. Herein, “proximate” and “proximately” can describe an atomic forcipes that is in or near a biological structure, including a neural structure. Additionally, provided are methods of employing the BMI using atomic forcipes, in which nuclear magnetic isotopes use their resonance coupling to achieve magneto-electric signal enhancement and antenna size reduction. Atomic forcipes can be a sensor, actuator, transmitter, receiver, or transceiver. Further atomic forcipes can be for example, and without limitation, a sensor/transmitter, an actuator/receiver, or a transceiver. Although the BMI will be described with respect to living brain tissue, it is to be understood that any junction in a living being can be populated with one or more atomic forcipes such that the atomic forcipes applications may be realized, for example, in nerve, bone, or muscle tissue.

For embodiments involving living biological tissue, significant molecular dipole orientation of water at or near radio frequencies of about 2.5 GHz for pure water and about 2.1 GHz to about 2.5 GHz for brain tissue can be avoided, where destructive thermal heating of living tissue occurs. Between about 10 KHz to about 1 GHz, the dielectric permittivity of brain tissue decreases, and conductivity increases as a power law of frequency. Above 1 GHz, an increasingly undesirable result is a conductivity increase that is quadratic with respect to the applied frequency due to dipolar reorientation of free water molecules, such that a very high and continuous radio power in this range is to be avoided as not applicable to body-machine interface (BMI) embodiments. However, clinical trials for mammographies have shown a safe region of about 2 GHz to about 4 GHz exists for radio wave irradiation sent in short pulses useful for time domain reflectometry, thereby allowing low radio power applied to a body-machine interface in pulses to activate atomic forcipes, for example and without limitation, in brain tissue, at this microwave frequency range.

The overall characteristics of selected embodiments herein make atomic forcipes uniquely suited to their application as a bidirectional body-machine interface (BMI), and in particular, a brain-machine interface, because the atomic forcipes can be implanted in neural tissue in vivo. Furthermore, in the context of body-machine interface, atomic forcipes can be configured as an interface to biological structures, including without limitation skeletal joints, muscle, and soft tissue structures. Just as any antenna can act as both a transmitter and a receiver, the same is true of atomic forcipes, which can be bidirectional transceivers. In the case of neural structures, the ingress of sodium or potassium atoms from the extracellular fluid into the atomic forcipes gallery provides immediate alteration of capacitive reactance as well as inductive reactance in the electrical operation and time response characteristic of the atomic forcipes, thereby making atomic forcipes a very sensitive frequency-based resonant oscillator sensor of the ionic concentration and, therefore, the hyperpolarization or the depolarization of a proximate neuron. Atomic forcipes can be sensors, placed proximate to a synaptic cleft, and can be used to monitor sodium ions in the synaptic cleft region of somatic neurons, and of glial interaction zones having a calcium ion release among glia interacting at the tripartite neurons of brain tissue. Glia, in general, maintain the ionic milieu of nerve cells, modulate the rate of nerve signal propagation, modulate synaptic action by controlling the uptake of neurotransmitters, provide a scaffold for some aspects of neural development, and aid in (or prevent, in some instances) recovery from neural injury. Atomic forcipes may also be used to activate calcium voltage-gated ion channels, and sodium voltage gated ion channels. When present proximate to a neural synapse, atomic forcipes can be used to initiate a transient neural transduction voltage spike signaling event, especially when dynamically activated by the directed energy of radio frequency or phonons.

One embodiment utilizes the nuclear magnetic isotope effect (MIE) to enhance the bidirectional wireless information sending and wireless receiving of atomic forcipes as a sensor and as an actuator used for communication by enhanced electromagnetic interaction as body machine interfaces (BMI). The atomic forcipes BMI can be implanted, inter alia, in neural tissue, in vivo. Graphene is biocompatible, and aluminum magnesium silicate such as montmorillonite is well tolerated with antiviral characteristics in biological tissues. The atomic forcipes function as piezo-electrically driven resonators with different particle or sheet sizes having both amplitude and phase modulation of electric fields, which can be read out independently and simultaneously in their production of radio waves emanating from an abutting conductive graphene sheet producing corresponding ratios of amplitude and phase modulations. To further increase the number of signals for the combination, different mechanical modes of individual atomic forcipes are used in parallel, since there is no coupling between non-abutting atomic forcipes. The desired RF irradiation frequency can be in the Terahertz (THz) range, but also may be in the Megahertz (MHz) range to avoid heating of biological tissues. Ultrasonic actuation frequency can be about 80 Kilohertz (KHz) to reduce the size of potentially destructive cavitation and to increase penetration and actuation of atomic forcipes. Current neural interface devices are designed to perform a single function: either to record neural activity or to electrically stimulate neural tissue, where either function requires a different device. The atomic forcipes sets a unique example as the first BMI device to integrate both functions into one device structure, wherein are provided two alternative types of irradiative actuation to enable external neural transmission or to enable wireless reception to obtain the neural polarization status, transient ionic characteristics, and electrical spike firing condition of neurons.

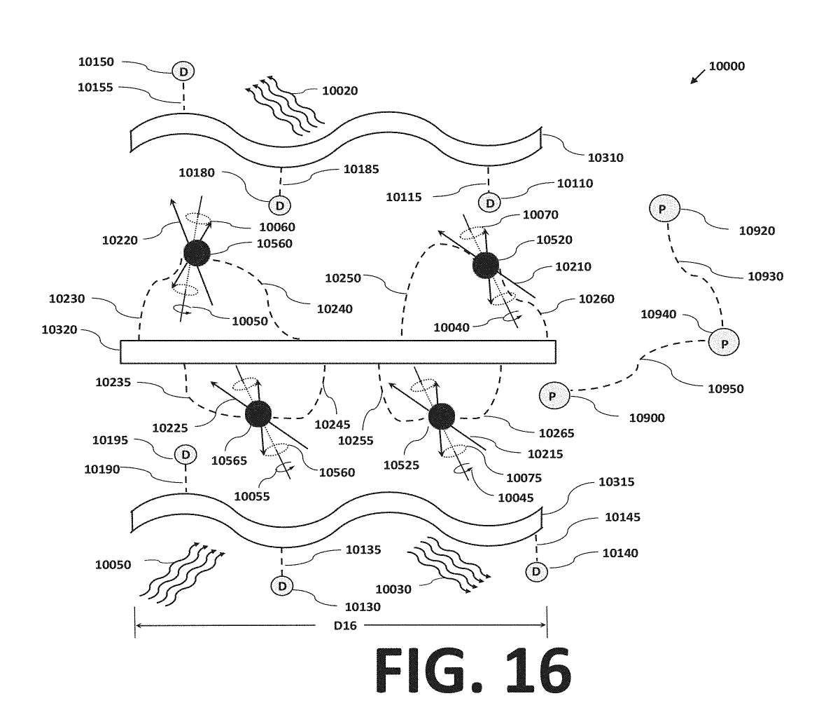

FIG. 16 shows an embodiment of a neural atomic forcipes probe including two deuterated ferromagnetic graphene sheets 10310, 10315 in abutment to clay sheet 10320. The clay sheet 10320 can have multiple exemplary 57-Fe nuclear magnetic ions 10520, 10560, 10525, 10565 bonded adducts, which each have nuclear magnetic moments 10210, 10220, 10215, 10225 and electric moments 10040, 10050, 10045, 10055 affecting their precession 10060, 10070, 10065, 10075 coupled to their electromagnetic domain formation to assist in the attraction of electron charge carriers. The electron charge carriers may otherwise interfere with the ballistic Dirac electrons in graphene sheets 10310, 10315 used for resonance with externally applied and re-radiated Terahertz radio frequencies. Multiple transiently conductive protein molecules 10900, 10920, 10950 can form a voltage carrying network along 10930, 10950 that will supply electric current to the exterior of graphene sheets 10310 10315. This current can affect the charge and therefore the distance between graphene sheets 10310 10315 and the clay sheet 10320. Nuclear magnetic isotopes 10560, 10520, 10565, 10525, selected to be ferromagnetic and having adduct bonds 10230, 10240, 10250, 10260, 10235, 10245, 10255, 10265, can provide ferromagnetic coupling with multiple pendant bonded deuterium atoms 10180, 10110, 10195, 10150, 10130, 10140 through bonds 10155, 10115, 10190, 10145, 10815, 10135 causing ferromagnetic properties in graphene sheets 10310, 10315. This decreases the conductivity and increases the permittivity component of applied Terahertz radio frequencies or infra-red-light irradiation indicated by 10050 to shift and modulate energy that will be absorbed and then re-emitted as 10030 and 10020. Sodium ions having greater than about 113 GHz loss component absorption, will remain approximately constant, for example, in the region outside the neural synaptic cleft, whereas the voltage gradients will be subject to change. Multiple chemical-adduct nuclear magnetic isotopes 10520, 10560, 10525, 10565 may be selected from any element of odd numbered atomic mass, having one more neutron than the count of protons, to enable expression of coupling between their atomic electric field and the nuclear magnetic precession. The neural atomic forcipes of size D16, being greater than about 30 nanometers to about 500 nanometers, will have sufficient directional dielectric properties to act on the energy of externally broadcast electromagnetic irradiation of radio frequencies or infra-red light. However, a metallized back gate as illustrated in FIG. 15 may be applied to one of the external surfaces of one of the graphene sheets as needed. Generally, the resulting structure functions to generate a modulation associated with the voltage of local neural firing potentials expressed as neural electric charge, where this modulation information can be emitted from the neural atomic forcipes in an altered energy format to be remotely and wirelessly received.

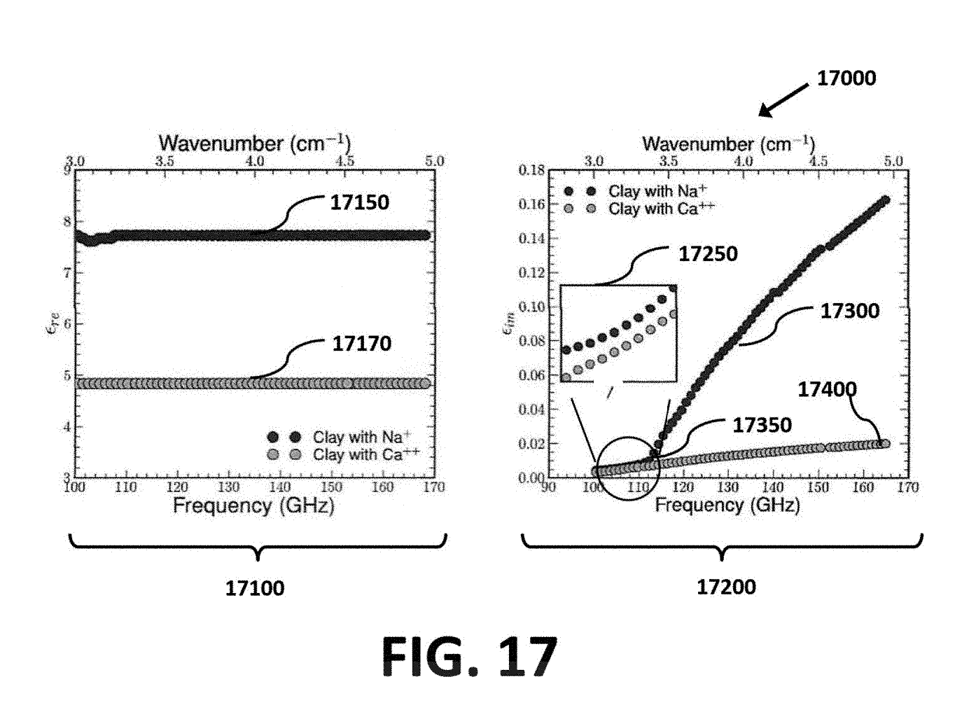

Referring now to FIG. 17, there are shown graphically the measured dielectric permittivity data values 17000 obtained for both smectite montmorillonite clay intercalated with calcium, and for smectite montmorillonite clay intercalated with sodium. The dielectric storage permittivity for calcium intercalated clay and sodium intercalated clay is shown in graph 17100 as being constant with frequency but different in value. This value is higher at 17150 for sodium than it is for calcium at 17070, thereby providing AC electromagnetic information about the gallery ions being exchanged with the ambient ions of the fluid environment in which these clay particles are suspended. The dielectric permittivity loss is shown in graph 17200, wherein a subset of that data circled at 17350 is magnified and shown as inset 17250 indicating a region of no significant deviation between calcium and sodium loss permittivity below 113 Gigahertz. A significant nonlinear deviation in loss permittivity of sodium intercalated clay is shown by curve 17300 as compared with the curve for calcium intercalated clay 17400 for frequencies of irradiation greater than 113 GHz. This indicates high sodium ion mobility imparted as physical motion that has been induced by these frequencies of irradiation. Therefore, sodium ions will be not only electromagnetically jostled, but also expelled from the galleries when sodium containing clay is subjected to irradiation greater than 113 GHz at significantly high field intensity, while calcium ions will remain relatively unperturbed at these frequencies.

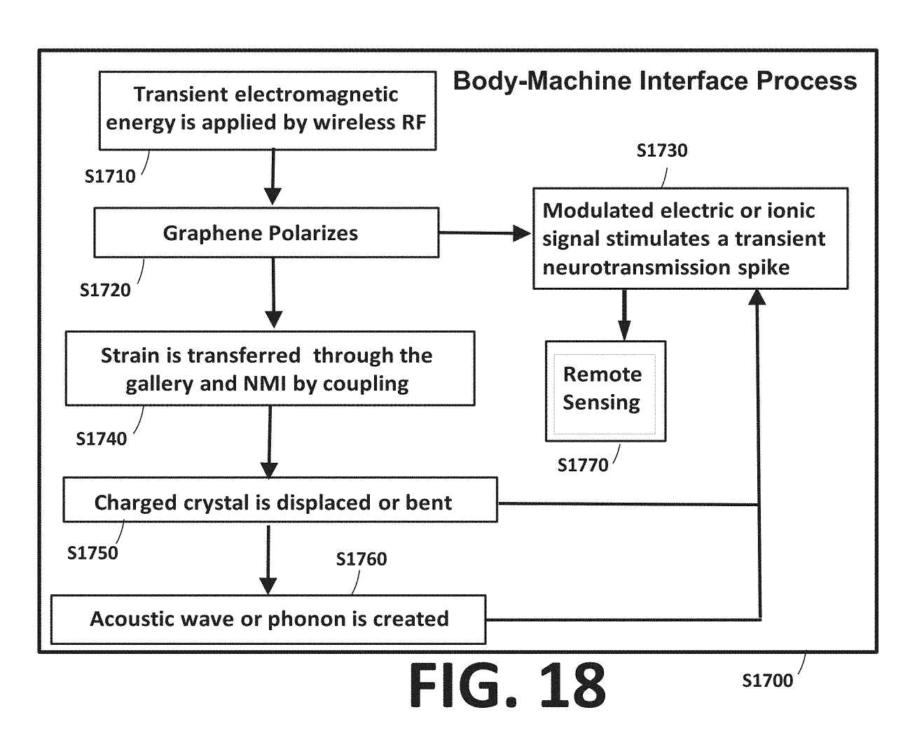

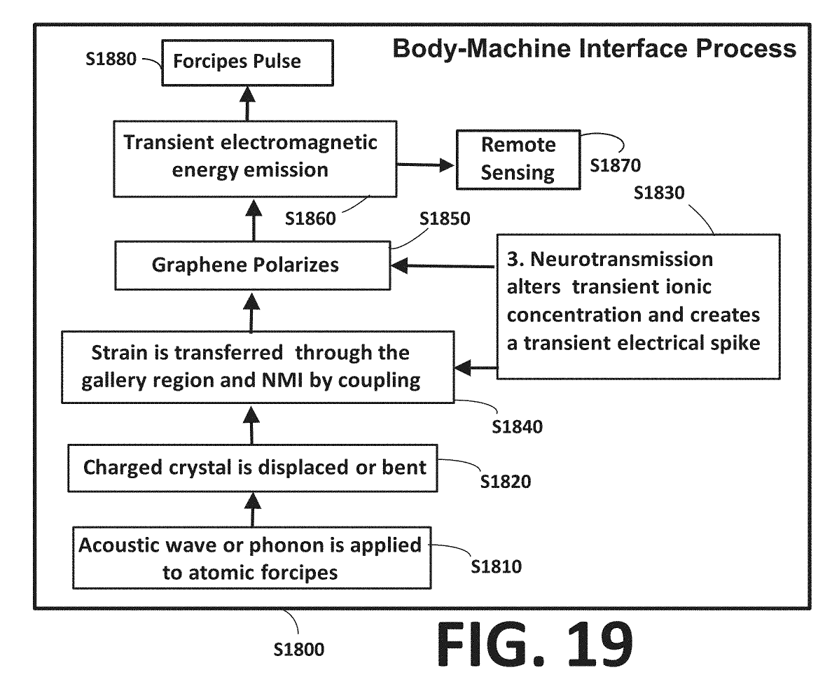

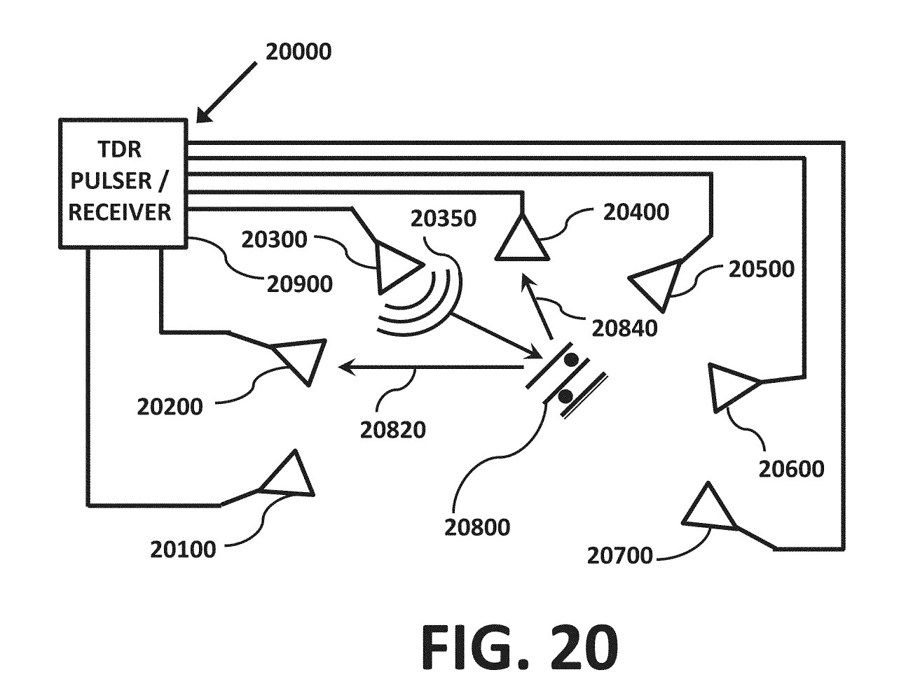

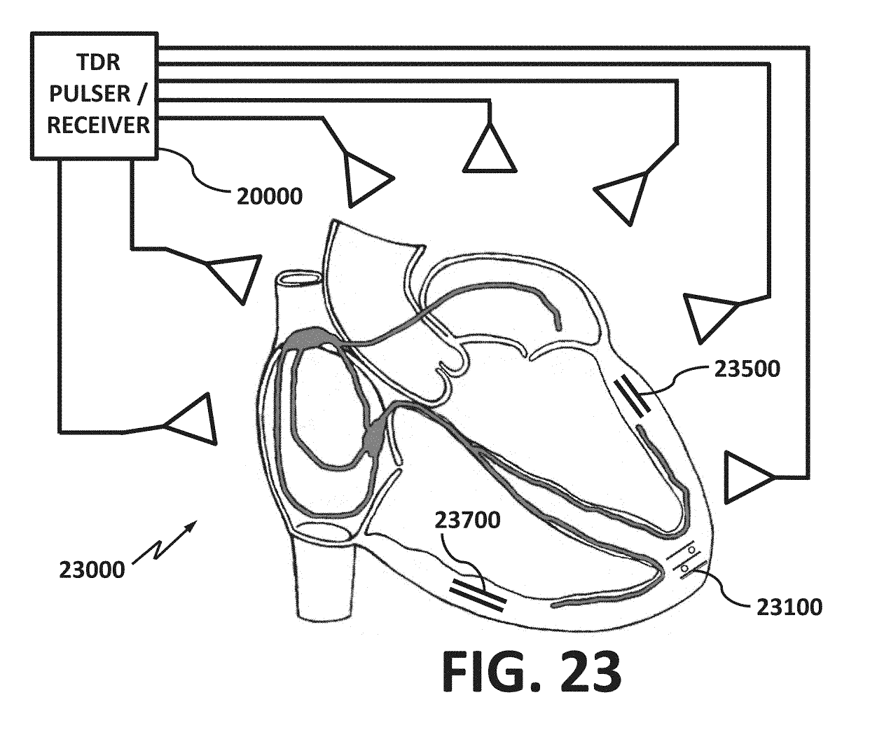

Referring now to FIG. 18, there is shown a body-machine interface process (S1700) using atomic forcipes, such as atomic forcipes 9000, proximate to the neural spaces as well as proximate to living neurons, as shown in detail in FIG. 21, and also shown in the context of a human brain in FIG. 23. The atomic forcipes of step S1710 can be between about 11 nanometers to about 25 nanometers in diameter, and are provided with deuterated ferromagnetic graphene sheets, abutted to and interposed with, clay sheets having intercalated adducts composed of nuclear magnetic isotopes (NMI), for deliberate interaction with an applied radio frequency pulse supplied by an external source, such as shown in FIG. 20. In step S1720, the received electromagnetic energy supplied as a pulse of light or radio waves acts on the deuterated ferromagnetic graphene conductor sheets to apply a time-varying polarizing voltage to the atomic forcipes. This voltage conducts in part to the gallery containing chemically bound nuclear magnetic isotopes, and mixes electromagnetic energy with mechanical atomic displacements (phonons) to create a plasmon. The plasmon is confined to the gallery and is a hybrid of mechanical and electromagnetic energy interactions. This plasmon creates charge carrier migration among free ions, especially expelling sodium ions from the gallery into the synaptic cleft as now the piezoelectric clay sheet component of the atomic forcipes responds to the presence of a polarizing voltage and moving charges to induce mechanical acoustic vibration in the form of generated phonons (sound waves) by electromagnetic interaction. This acoustic emission enhances diffusion of any atoms or compounds in the vicinity of the atomic forcipes, including that of neurotransmitters normally released into the synaptic cleft environment. In addition, the polarizing voltage generated by the atomic forcipes may add to the local potential required to initiate a neural electrical transmission discharge, also known as a neural electrical spike. Each of these contributions then leads to step S1730, neural transmission initiation of an electrical signal. Expression of a voltage spike of about 50 millisecond duration then substantially alters the mechanical vibration frequency of the atomic forcipes for this duration. This alters the gap distance and the capacitance of the atomic forcipes for a time such that any future absorbed electromagnetic pulse provided by a remote transmission results in altered atomic forcipes electromagnetic emissions. These emissions can be recorded by external signal monitoring of this neural spiking event, for example, by a computer that includes the TDR pulser and receiver shown in FIG. 20. Independent of step S1730 is the continued voltage-mediated activation of the atomic forcipes of type 9000 displacement in step S1740 where the strain energy carried through the gallery becomes modified as the mobile ion concentration is altered in the galleries of this device by fluid transport and ionic diffusion directly related to the ions present in the immediate extra-neural space of this device. For example, potassium ions will enter into the gallery and change the atomic forcipes current and voltage characteristics. These effects now produce a characteristic shift in the amount that the piezoelectric sheet is bent in step S1750, which alters the voltage expressed by that material, and this voltage alteration alters the device capacitance and permittivity signals both in magnitude and frequency in step S1760 wireless monitoring dependent on the identity of the newly injected ions and their concentration, both of which inform on the state of hyperpolarization or depolarization of the nearest neuron having emitted such ions as a result of neural processes. S1770 indicates remote sensing of the neuron polarization status, for example, by Fourier Transform analysis of any returned electromagnetic emission pulse during this period. Fourier transform analysis of the time-domain reflected electromagnetic pulse is used by controller 20000 in FIG. 20 to receive and decode atomic forcipes information as described with respect to FIG. 19.

Referring now to FIG. 19, there is shown a body machine interface process S1800, different from the body machine interface process S1700 in FIG. 18, using atomic forcipes of greater than about 1000 nanometer that have been implanted in between or abutting the extra-cellular spaces of the living tissue of body organs for the purpose of actuating them. The atomic forcipes are constituted of deuterated ferromagnetic graphene sheets that have piezoelectric clay sheets interposed between them. Note the environment of the micron sized atomic forcipes, i.e., recurrent loads or acoustic irradiation, is not a necessary process, but it is certainly a measurable one that may assist with signal analysis in a post-processing of Fourier transformed frequency and time information. Triangulation is described with respect to FIG. 20, and is another optional analysis rather than a necessary process. Fourier transform analysis of the time-domain reflected electromagnetic pulse is used by controller 20000 to receive and decode atomic forcipes information. Note that both particle size and NMI identity are used to configure the function of the process of FIG. 19. The atomic forcipes of step S1810 are provided with intercalated adducts composed of nuclear magnetic isotopes for resonant interaction with electromagnetic irradiation applied to the tissues from an external pulse generator 20000. In one embodiment, intercalated ferromagnetic iron cations Fe+2 can be provided where the positively charged ferromagnetic iron cations are the nuclear magnetic isotope 57-Fe. In another embodiment, intercalated manganese cations Mn+2 can be provided where the positively charged cations are the nuclear magnetic isotope 55-Mn. In yet another embodiment, intercalated copper cations, Cu+2, can be provided where the positively charged cations are a mixture of the nuclear magnetic isotopes 63-Cu and 65-Cu in an approximate ratio of 69:31 by atomic mass weight to atomic mass weight, respectively. In yet another embodiment, intercalated Cobalt cations, Co+2, can be provided where the positively charged cations are the nuclear magnetic isotope 59-Co.

High field electromagnetic waves can be applied to energize those atomic forcipes resonating at the characteristic frequency induced by their component parts, especially as induced by the identity of their pure intercalated nuclear magnetic isotopes. Several of these NMI atomic forcipes embodiments are mixed to provide a gradient of excitation levels that is frequency pulse dependent as well as amplitude dependent. This energization is partly expressed as a polarization of the graphene sheet, and partly expressed as phonons and plasmons that result in the dynamic displacement and bending of the piezoelectric component of the atomic forcipes in step S1820; the oscillating and frequency dependent deformation of the piezoelectric sheet causes time varying voltage to appear. The strain energy and the voltage act on the bonded nuclear magnetic isotopes as well as the abutting conductive graphene sheet to induce current flow and transient polarization in step S1850. However, another source of current flow may arise in the abutting neural tissue in step S1830 that transfers ions into the atomic forcipes gallery and acts to modify the voltage signal transduction and therefore also the amount of strain transferred through the gallery in step S1840. This changes the time varying voltage expressed as a current in the polarization of graphene in step S1850. Any time varying currents produced in step S1850 produce a wireless radio wave transmission that is capable of being received at some remote distance for recording of amplitude and frequency changes associated with the electrical state of the nearest neurons, dependent on the identity of the newly injected ions and their concentration, both of which inform on the state of hyperpolarization or depolarization of the nearest neurons having emitted such ions to control the actuation of muscular contraction processes. Triangulation is described in FIG. 20 and is an optional analysis useful for correlating the analytical response of the muscle to a located signal source for each embodiment type of atomic forcipes by Fourier transform analysis of the returned pulse in S1870. This information is required to decode atomic forcipes neuron polarization status information at sufficiently high field strength for a pacemaker level of cardiac muscular contractions. In the case of a multiple, electrically percolating, and greater than 1 micron sized atomic forcipes, a sufficiently high field electromagnetic pulse may result in S1880 to provide adequate voltage expressed by atomic forcipes for defibrillation.

Referring now to FIG. 20, there is shown a commercially available multichannel programmable time domain reflectometry (TDR) system 20900 for remote control broadband pulse generation and Fourier transform processing of the returned signals, which may be configured for use with one or more types of atomic forcipes. Frequency domain reflectometry also may be used, mutatis mutandi. Exemplary atomic forcipes 20800 may be composed of multiple smaller atomic forcipes and may additionally be composed of differently intercalated types of atomic forcipes wherein such atomic forcipes may be a mixture or stratified across organs and tissue types to locally allow a separation in the placement of activation or sensing modalities. Such atomic forcipes 20800 may be applied topically or implanted, for example, to operate as a source of voltage, or to operate as a source of heat applied to tissues. Multiple antennas 20100, 20200, 20300, 20400, 20500, 20600, 20700 are configured by 20900 to receive electromagnetic energy in the form of radio waves in the microwave (gigahertz or GHz) band or terahertz (THz) band, or configured to receive light waves where any of these may be composed of light such as far infrared frequencies that may also be modulated in the terahertz (THz) band, especially in the form of a reflected pulse of irradiation. In selected embodiments, optical chirp or RF chirp pulses may be used. Near infrared pulses on the order of picosecond to femtosecond durations can be an example of such pulses. In some system embodiments, there may be more or fewer antennas than shown, depending on the number of channels for the commercial unit. Each antenna used for reception may be configured or programed to act as an antenna. One antenna is normally reserved for the generation of one pulse at one time. The direction and orientation of multiple reception antennas assures a volume of space can be used to receive emission and reflection radiation from irradiated atomic forcipes 20800 from any direction. Exemplary antenna 20300 is shown in FIG. 20 to be the one that is momentarily configured to supply an irradiative and preferentially coherent broadband pulse 20350 that propagates in a direction toward 20800 as indicated by the arrow arising from electromagnetic waves 20350. The initial time of the pulse is recorded by the TDR pulser 20900. This preferentially coherent irradiation pulse 20350 is absorbed by atomic forcipes 20800 optionally having a backplane as illustrated in FIG. 15 to assist in directed re-emission of radiation towards multiple antennas including 20200 and 20400. The time of the pulse reception is recorded by TDR receiver 20900 as each antenna obtains a signal response in elapsed time that is measured by difference from the time of the initial pulse irradiation time, and this duration will depend on the distance from the atomic forcipes 20800, where greater distances provide longer durations. Fourier transform analysis is then performed on the received waveform for each antenna to seek frequency broadening effects and to report on the creation of sidebands or absorbance line shifts compared to the ideal linewidth expected from nuclear magnetic isotopes in atomic forcipes 20800 used for external calibration reference that are by comparison not exposed to biological tissues, or fluids, or pressures that would otherwise serve to alter the absorbance resonance modes of atomic forcipes devices.

Referring now to FIG. 21, there is shown an irradiated neural structure 21000 including a biological transmitting neuron 21710, a biological receiving neuron 21700, and a synaptic cleft 21100. Biological neural transmission information is shown to be sent and received by artificial wireless transmission using atomic forcipes 9000, 10000 fitted or placed proximate to (in or near) synaptic cleft 21100 to act as a wireless transceiver of the neural information. Atomic forcipes 9000, 10000 in FIG. 21 may be structurally and functionally equivalent to atomic forcipes 10000 in FIG. 16. Atomic forcipes 9000, 10000 can be used to wirelessly monitor and report neural activity to a remote-control station 20000 capable of producing electromagnetic pulses for time domain reflectometry (TDR) and receiving the echo of such pulses for triangulating the distances to the reflecting atomic forcipes by means of at least two reception antennas as illustrated in FIG. 20. Atomic forcipes, particularly atomic forcipes 9000, can be used to selectively impart actuation signals into the synaptic cleft, causing the neuron to fire, by releasing sodium atoms into the cleft, and by producing a small amount of voltage in approximately nano-voltage and nano-amperes of current intensity, as a result of being actuated by an electromagnetic pulse of sufficient intensity.

The primary electrical and chemical signals necessary for somatic control and biological neural processing are transmitted from, for example, 21700 to 21720 through the synaptic gap 21100. Secondary moderating signals arise from chemical exchange of proximal calcium+2 ions (herein Ca+2) at 21240 and 21260, however Ca+2 at 21250 is also present inside the signal sending axon 21700 gated through Ca+2 ion channels 21310, 21320, 21330 transferring Ca+2 ions outside to extra-neural fluids at 21200, 21220. The membrane potential of the individual neuron at rest is about −70 mV. Signal sending axon 21700 contains neurotransmitter molecules in vesicles 21500, 21520, 21540 which migrate to the region of the synaptic cleft 21100 at multiple porous regions 21900 for release prior to electrical signal transduction, where these molecules will bond to the exterior exposed surface structures of multiple sodium gated ion channels 21600, 21620, 21640, 21650, 21660, 21680 where this bonding serves to open the sodium ion gates to allow passage of charge carrier sodium ions (herein Na+) 21800, 21820, 21840, 21860 from the receiving neuron 21710 into the synaptic cleft 21100 to allow natural biological electrical and chemical neural signal transduction to take place. The neural structure has been implanted with multiple like atomic forcipes 9000 to allow measurement of Na+ ions 21820, 21840, 21860 released by the receiving axon 21710. Radio frequency activation of the multiple atomic forcipes 9000 enables enhanced mixing of neurotransmitter molecules and ions in the region of the synaptic cleft 21100, by means of induced flexural deformations of atomic forcipes giving rise to acoustic phonons that act to mechanically agitate and mix the synaptic fluid, thereby increasing the likelihood of neural signal triggering.

The wireless reporting function of the atomic forcipes in these neural structures can emit electromagnetic energy arising from strongly coupled ionic and electrical signals at the region of neural membrane as the neuron undergoes the biological polarization and depolarization process. Initially, the depolarized resting state of the neural membrane expresses a potential of −70 mV at transmitting neuron 21700. This potential will begin to alter as neighboring neurons (not shown) supply it with excitatory (depolarizing) and inhibitory (hyperpolarizing) voltage spikes originating from other, even more distant neurons in the body (not shown). Given a sufficient cumulative amount of such voltage spike input, an action potential will be generated just prior to the point where the transmitting neuron 21700 will begin to transmit its own voltage spike across the illustrated synaptic cleft 21100 to the receiving neuron 21710. This begins to happen as the membrane potential of transmitting neuron 21700 surpasses a slightly more positive threshold voltage of about −55 mV in the presence of neurotransmitter molecules, where this polarization acts to open the voltage-gated channels 21600, 21620, 21640, 21650, 21660, 21680 that flood the neural synaptic cleft 21100 with multiple positively charged sodium ions 21800, 21840, 21860, providing a conductive path for voltage to pass as a transient voltage spike. Once this voltage has passed, a rapid depolarization of the neural synapse results as follows: The neural membrane potential will reach a voltage of about +30 to +40 mV indicating a state of complete depolarization, and the neural membrane of transmitting neuron 21700 will then begin to repolarize via the expulsion of positive charged potassium ions (herein K+) 21990 through potassium gated ion channels 21970, 21980 to restore a net negative charge condition that brings transmitting neuron 21700 back to relax to the resting potential of about (−70) mV. One function of atomic forcipes 9000 having a nominal diameter of about 11 to about 25 nanometers, is to wirelessly send and receive electromagnetic energy arising from strongly coupled ionic and electrical signals at the region of neural synaptic membrane, especially in the active region of the synaptic cleft 21100, to read the type of ions resident in that region based on atomic forcipes resonant frequency response. This function is enabled after an excitation introduced externally by an electromagnetic pulse arriving from the TDR pulser 20000. The characteristic decay time of the emitted signal from atomic forcipes will be altered when potassium ions 21990 intercalate into the gallery in place of sodium ions; these ions also contribute to a shifted electric loss response, and this combination of signals helps to determine the polarized or resting state of the neuron based on the ions near to and within the structure of the atomic forcipes. A second function of the atomic forcipes is to activate the opening of sodium ion channels by use of a sufficient high field energy pulse, wherein this will polarize the graphene component of atomic forcipes and also act to expel some of the intercalated ions; both the voltage development and the ionic expulsion act together to help activate the transmission of a voltage spike across the synaptic gap 21100. Neural states are sensed, and neural spike transmission is activated when atomic forcipes are used with a TDR pulse generator 20000.

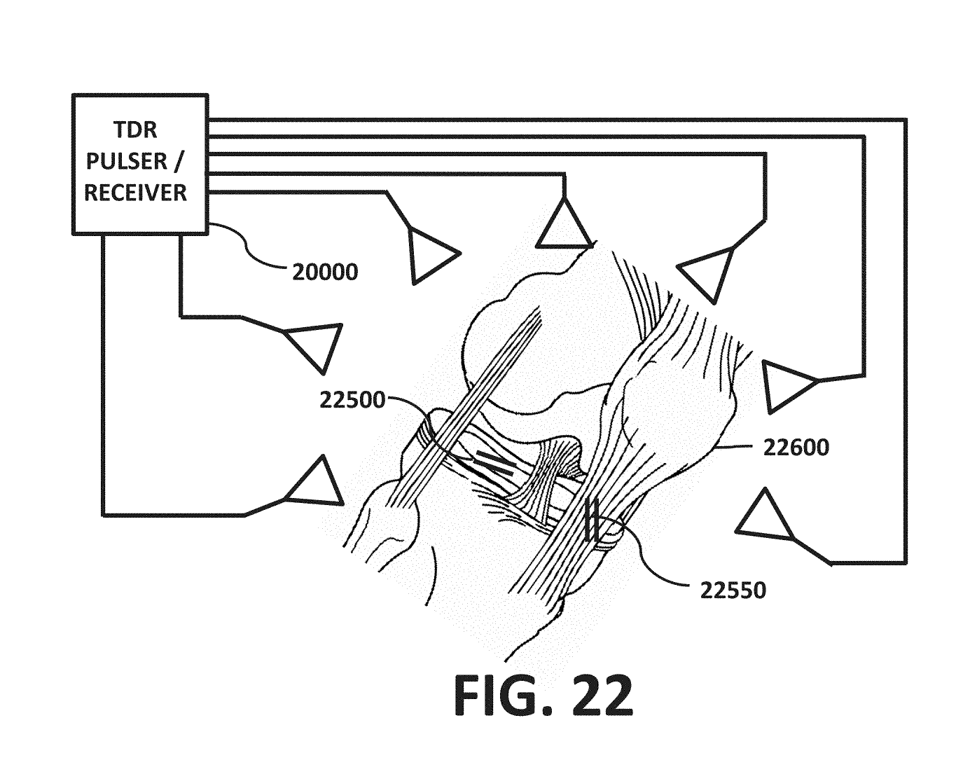

Referring now to FIG. 22, there is shown wireless atomic forcipes active implantable medical device (AIMD) to generate voltage for muscle actuation by voltage and ion mediated contraction at joint 22600. Atomic forcipes 22500, 22550 are configured for a pressure sensing function using a combination of long chained intercalated proteins illustrated in FIG. 10 and shown intercalated in FIG. 12. Native body fluids penetrate 22500, 22550 by seeping into atomic forcipes under conditions of pressure and pressure release at the site of use in a load bearing joint. Pressure on 22500, 22550 causes the atomic forcipes to deform by reducing the average gallery gap which serves as well to expel charged sodium ions; this combination of effects alters the atomic forcipes capacitance and the radio frequency response to report on local pressure changes in the joint. Atomic forcipes 22500 may consist of multiple atomic forcipes 22500, 22550 in high concentration to allow multiple electric contact points which alter their points of contact on the application of pressure. Some sodium ions will be expelled from atomic forcipes, for example, implanted in a bone joint as a result of compression between the bones of a load bearing joint. Changes in load will simultaneously change the dielectric loss permittivity of pressure sensing atomic forcipes. Commercially available soft medical hydrogels composed with cartilage or cartilage compatible soft polymers are often used for medical implantation purposes. Any of these commercially-available polymers and medical implants may be used as carriers of atomic forcipes to confer dynamic pressure sensing and wireless detection capability to such conventional medical implantations.

Referring now to FIG. 23, there is shown an active implantable medical device (AIMD) 23000 for handling high RF power, and for delivering typically periodic pulses to the myocardium. Atomic forcipes 23100, 23500, 23700 configured as transient voltage generators to supply pacemaker voltages stored in the manner of a capacitor and released to provide cardiac contractions. A defibrillator function also may be included. In general, living muscular tissue responds to electrical stimulation by contracting. Atomic forcipes 23100, 23500, 23700 can serve as a wireless receiver and an actuator, as well as a wireless transmitter and sensor, in muscular tissue, including the heart. In a cardiac pacemaking/defibrillating application, atomic forcipes 23100, 23500, 23700 can be configured to be a wireless receiver/actuator to apply an electrical impulse to cardiac tissue to promote proper pacing when so commanded by an external wireless pulser/controller TDR 20000, which has a transceiver and control circuitry therein. Atomic forcipes 23100, 23500, 23700 also can be configured to be a sensor/wireless transmitter to sense cardiac electrical activity and to send representations of cardiac electrical signals to the external wireless controller 20000. In this case, a different nuclear magnetic isotope is intercalated into 23500 than 23700 to confer a characteristic nuclear magnetic resonance absorption that can be converted into a voltage. However physiological sodium and calcium ions also reside in the gap regions of each of these atomic forcipes to expedite coupling to the cardiac tissues by means of ionic charge carriers. The size of atomic forcipes can be at least 1 micron. The concentration of these atomic forcipes is sufficient to provide electrical contact among the implanted atomic forcipes that are useful to supply sufficient current applied under a high field magnetic resonance imaging pulse (MRI) of the type that may also be used for time domain reflectometry (TDR). AIMD 23000 avoids the range of frequencies bounded by an upper frequency of 113 GHz to avoid sodium ion RF coupling modes where these ions become heated, mobile, and oscillate at high field strength radio frequencies. This effect was shown graphically in FIG. 19 at right for the sodium loss modulus represented by the upper dark marked curve 17300. The voltage generated at low loss follows the graphene polarization illustrated in FIG. 12, where at least one abutting clay sheet serves as the capacitor to store charge that is released by graphene into the surrounding tissues at exposed edges. A local voltage pulse into the surrounding tissues results once sufficient potential is expressed at the characteristic breakdown voltage of the cardiac tissue in which the atomic forcipes device or devices are implanted or to which they are affixed. Preferentially, two layers of clay, as shown in FIG. 12, can dissipate heat generated by high power radio waves. An optional metal back-plate, shown in FIG. 15, focuses electromagnetic energy into atomic forcipes 23100, 23500, 23700, and allows less energy to achieve significant voltage polarization on the graphene component of atomic forcipes. Typical cardiac pacemaker voltages of about 10 volts can be generated by atomic forcipes of about 1 micron or greater diameter, however several such devices can be used to enable the expression of sufficient milliamperes of current to achieve a physiological response without the generation of significant heat, depending significantly on the sites of placement, their distribution, and also on the extent of blood perfusion which alters the breakdown voltage of the cardiac tissues at that site. The use of multiple sequential pulses supplied by the external pulse generator is programmed to generate these pulses at different frequencies corresponding with the NMI intercalant of each type of atomic forcipes. These pulses elevate the total voltage and currents induced among the electrically percolating deposition at each site of implantation of atomic forcipes 23100, 23500, 23700 so that a sufficient voltage is supplied, for example, to enable defibrillation. Periodic stimulation can be used to provide regular stimulation, for example, to the heart. Intermittent stimulation can be used to selectively provide muscular contraction, for example, to cause movement of a paralyzed limb, to maintain muscle strength and tone during rehabilitation, or to an athlete to improve performance.

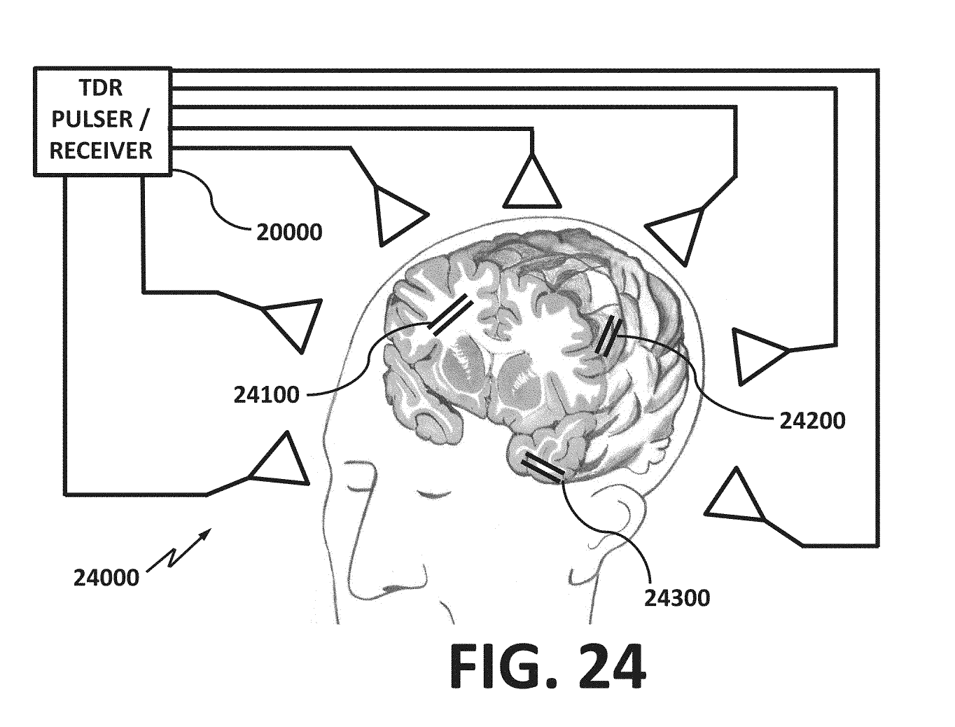

Referring now to FIG. 24, there is an active implantable neural device (AIND) 24000 for brain machine interfaces (BMI) in brain tissues. AIND 24000 employs regional implantation of atomic forcipes 24100, 24200, 24300 as sensors. Time domain reflectometric pulses are supplied by remote control device 20000 to actuate the neural status sensing of multiple implanted atomic forcipes at each brain region 24100, 24200, 24300. The nuclear magnetic isotope (NMI) of all of the multiple atomic forcipes at each region 24100, 24200, and 24300 are the generally the same at each such region. However, a different NMI is implanted at each region, so that the interaction of the different brain regions can be processed. This is useful in that during some types of brain activity, entire regions of the human brain undergo concerted neural activation associated with a specialized type of cognitive function, such as visual processing, or auditory processing, and the like, wherein each of the activated regions undergo a collective cyclic electric activation at a characteristic neural firing frequency. TDR performed using pulses targeting each type of NMI at the different resonant NMI frequencies can help to clarify the distribution of thought processes among and between each monitored brain region.

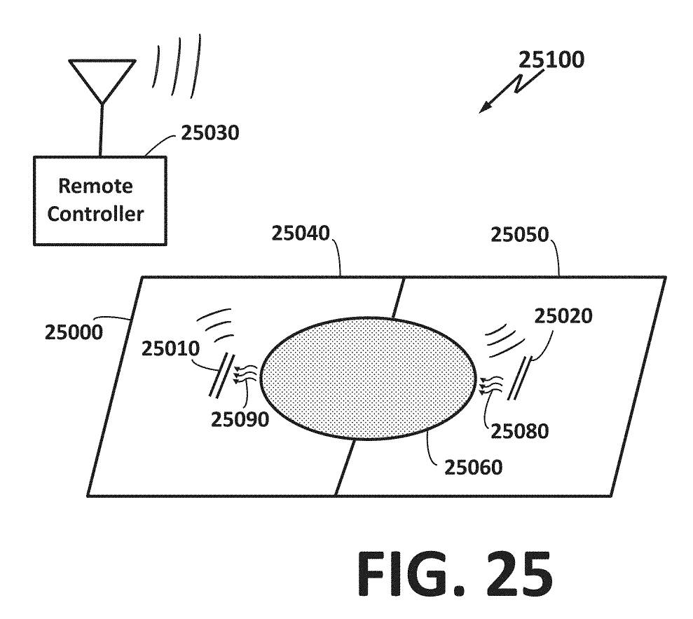

In FIG. 25, AIMD 25100 is illustrated to include atomic forcipes 25010, 25020, which are shown proximate to (in or near) a biological structure 25000, and remote controller 25030. Remote controller 25030 can be a medical magnetic resonance device configured to provide radio frequency pulses, which may be, for example, generally continuous signals. Remote controller 25030 also may be configured as a TDR pulser/receiver 20000, as in FIGS. 20-24. Biological structure 25000 can include, without limitation, bone, muscle, or soft tissue. Atomic forcipes 25010, 25020 can transceive biological structure information with biological structure 25000, for example, using remote controller 25030. Biological structure information can be physical characteristic representations, which include, without limitation, physiological, biochemical, bioelectrical, and biomechanical parameters. Atomic forcipes 25010, 25020 can be used to sense and report one or more biomechanical parameters in structure 25000 including, without limitation, load, pressure, torque, shear, stress, strain, tension, compression, flexion, distension, or extension. Atomic forcipes 25010, 25020 also can be used to sense and report one or more bioelectrical parameters in structure 25000 as well including, without limitation, voltage, current, trans-epithelial potential, plasma membrane voltage gradient, charged biomolecule movement, diffusion, capacitance, ionic composition, ionic concentration, or ionic flow. Physiological parameters sensed and reported by atomic forcipes include, without limitation, temperature, respiration, pulse rate, and blood pressure, for example, in the region of the atomic forcipes 25010, 25020. Biochemical parameters include, without limitation, biomolecule presence, absence, composition, or concentration, for example, of a protein.

Atomic forcipes 25010, 25020 can be a sensor, generating a representation 25090 of biological structure information, and can be a transmitter, reporting the representation to remote station 25030. Moreover, atomic forcipes 25010, 25020 can be a receiver, receiving information for use by structure 25000, and be an actuator, by which biological structure 25000 can receive a stimulus 25080 from external control station 25030. Atomic forcipes 25010, 25020 can be a transceiver having a length of between about 5 nanometers to about 10 microns, and be configured to transceive information with biological structure 25000. Therefore, AIMD 25100 can be used to monitor physiological, biomechanical, or bioelectrical parameters pertaining to biological structure 25000.

Structure 25000 can include first portion 25040 and second portion 25050. Structure 25000 also may have intermediate 25060 disposed between first portion 25040 and second portion 25050. Intermediate 25060 may be, for example, representative of any joint in the body, whether in an upper extremity, a lower extremity, or the spine. Thus, when intermediate 25060 is a joint, atomic forcipes 25010, 25020 can sense and report one or more biomechanical parameters in one or more of intermediate 25060, or in first portion 25040 or second portion 25050, with respect to intermediate 25060. The one or more physiological, bioelectric, or biomechanical parameters may be representative of biological structure 25000 state (e.g., joint configuration). Atomic forcipes are not limited to use in natural joints, but also may be used in prosthetic apparatus, mechanical replacement joints or in bone wounds, for example, to monitor damaged bone repair.

Additionally, atomic forcipes 25010, 25020 can be used as a sensor/transmitter disposed in soft tissue intermediate 25060, such as, without limitation, the skin, a soft tissue organ, a tumor (such as a melanoma), or a surgical or traumatic wound. In this application, atomic forcipes 25010, 25020 monitors soft tissue intermediate 25060 milieus, reporting back on, without limitation, physiological, bioelectric, or biomechanical parameters, fluid movement, fluid accumulation, tissue composition, tissue growth or necrosis, infection, post-trauma wound repair, wound dehiscence, and soft tissue intermediate 25060 milieu compositions (indicated, e.g., at 25090). Moreover, when used as a receiver/actuator, atomic forcipes 25010, 25020 may provide mechanical, electrical or electrochemical stimulation 25080 of soft tissue intermediate 25060 as part of AIMD 25100. Mechanical stimulation 25080 may include stimulation by acoustic phonons. Electrical stimulation 25080 can include the guided transfer of electrons into soft tissue intermediate 25060, as in an applied electrical current, for example, to encourage wound healing. Electrochemical stimulation can include, without limitation, infusion of preselected nuclear magnetic isotopes (NMI) into soft tissue intermediate 25060 when atomic forcipes 25010, 25020 are so configured. Infusion of NMI can be used in medical imaging, as well as in an attack on a cancerous cell or tumor. In another application, atomic forcipes may be used in a topical cream, for example, to treat a malignant melanoma, which may be shown as biological intermediate 25060. Surrounding healthy dermis may be first portion 25040 and second portion 25050. Atomic forcipes 25010, 25020 can magnify the THz signal responses by electromagnetic reaction with adsorbed biomolecules of surface skin to seek biochemical differences indicating melanomas that increase the signal differences far beyond what is achieved directly in THz irradiation.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but defined in accordance with the foregoing claims appended hereto and their equivalents.

1. A metamaterial structure, comprising:
a topological conductor having a preselected concentration of deuterons as chemical adducts therein;
a topological insulator expressing a net negative surface charge and having paramagnetic properties, and abutting the topological conductor; and
a gallery between the topological conductor and the topological insulator, the gallery having charged intercalated ions,
wherein an atomic forcipes is formed.

2. The metamaterial structure of claim 1, wherein the topological conductor comprises plural deuterated ferromagnetic graphene sheets of atomic layer thickness.

3. The metamaterial structure of claim 2, wherein the topological insulator comprises a clay sheet disposed between the graphene sheets.

4. The metamaterial structure of claim 3, wherein the atomic forcipes further comprises:
a preselected nuclear magnetic isotope disposed in the gallery and formed as an adduct to the clay sheet.

5. The metamaterial structure of claim 4, wherein the atomic forcipes comprises:
a wireless interface with a neuron.

6. The metamaterial structure of claim 4, wherein the atomic forcipes comprises: a receiver.

7. The metamaterial structure of claim 4, wherein the atomic forcipes comprises: a transmitter.

8. The metamaterial structure of claim 4, wherein the atomic forcipes comprises: a transceiver.

9. The metamaterial structure of claim 4, wherein the atomic forcipes comprises: a sensor or an actuator.

10. The metamaterial structure of claim 6, wherein the atomic forcipes has a length of between about 5 nanometers to about 20 nanometers, and is configured to wirelessly receive electromagnetic radiation from a neuron.

11. The metamaterial structure of claim 6, wherein the atomic forcipes has a length of between about 5 nanometers to about 20 nanometers, and is configured to wirelessly receive acoustic radiation from a neuron.

12. The metamaterial structure of claim 7, wherein the atomic forcipes has a length of between about 5 nanometers to about 20 nanometers, and is configured to wirelessly transmit electromagnetic radiation to a neuron.

13. The metamaterial structure of claim 8, wherein the atomic forcipes comprises: a transceiver having a length of between about 5 nanometers to about 20 nanometers, and configured to transceive electromagnetic radiation with a neuron.

14. The metamaterial structure of claim 8, wherein the atomic forcipes comprises: a transceiver having a length of between about 5 nanometers to about 10 microns, and configured to transceive information with a biological structure.

15. The metamaterial structure of claim 14, wherein the transceiver is a phase modulated transceiver.

16. The metamaterial structure of claim 14, wherein the transceiver is an amplitude modulated transceiver.

17. A body-machine interface (BMI), comprising:
a biological structure; and
atomic forcipes disposed in or on the biological structure, the atomic forcipes including:
plural deuterated ferromagnetic graphene sheets of atomic layer thickness,
a clay sheet expressing a net negative surface charge, having paramagnetic properties, and disposed between and abutting the plural graphene sheets, and
a gallery between each graphene sheet and the clay sheet, the gallery having preselected nuclear magnetic isotopes disposed in the galleries and formed as adducts to the clay sheet, wherein the atomic forcipes bidirectionally transceives information with the biological structure.

18. The BMI of claim 17, wherein the atomic forcipes transceive at least one of an acoustic signal or an electromagnetic signal, corresponding to one of an ionic signal or an electrical signal at a portion of the biological structure.

19. The BMI of claim 17, wherein the biological structure further comprises:
a neural structure having a sending axon terminal, a receiving axon terminal, and a synaptic cleft therebetween, the portion of the biological structure is the synaptic cleft, the atomic forcipes being disposed proximate to the synaptic cleft and bidirectionally transceiving information traversing the synaptic cleft.

20. The BMI of claim 18,
wherein the atomic forcipes comprises a nanomechanical magneto-electric (ME) antenna,
wherein the ME antenna receives oscillating electromagnetic (EM) waves,
wherein oscillating EM fields of the oscillating EM waves act to induce an oscillating electric field in the conductive graphene sheet of the ME antenna,
wherein the oscillating electric field induces an oscillating electric voltage across a substantially in-plane longitudinal aspect of the graphene sheet,
wherein induced electric field oscillations react against a static electric field of abutting piezoelectric material,
wherein mutually attractive and mutually repulsive mechanical forces arise between the abutting parts of the atomic forcipes,
wherein the mechanical forces oscillate in proportion to the induced fields to create phonons,
wherein the ME antenna comprises an RF activated ME antenna.

21. The BMI of claim 18,
wherein the atomic forcipes comprises a nanomechanical magneto-electric (ME) antenna,
wherein the atomic forcipes are acoustically-actuated,
wherein acoustic actuation further comprises: sonic waves provided to the atomic forcipes to stimulate magnetization oscillations in the graphene sheet of the atomic forcipes,
wherein the sonic waves have a frequency of between about 20 Hz to about 2.0 GHz, and
wherein the magnetization oscillations result in the radiation of electromagnetic waves from the ME antenna.

22. The BMI of claim 18,
wherein the atomic forcipes comprises a nanomechanical magneto-electric (ME) antenna,
wherein the atomic forcipes are electromagnetically actuated,
wherein the electromagnetic actuation further comprises: electromagnetic waves provided to stimulate electromagnetic oscillations in the graphene sheet of the atomic forcipes, wherein the electromagnetic waves have a frequency of between about 2 Hz to about 500 THz, wherein the electromagnetic oscillations result in the radiation of phonons.

23. The BMI of claim 18, wherein the atomic forcipes is a sensor proximate to the synaptic cleft, wherein the sensor detects a change in an ionic concentration in the synaptic cleft.

24. The BMI of claim 23, wherein the atomic forcipes further comprises a transmitter configured to transmit a representation of a neural state to an external device, corresponding to the change in the ionic concentration in the synaptic cleft.

25. The BMI of claim 23, wherein the atomic forcipes further comprises a receiver configured to receive a signal which initiates the change in the ionic concentration in the synaptic cleft.

26. The BMI of claim 18, wherein the atomic forcipes comprises a sensor proximate to the synaptic cleft, wherein the sensor detects a change in an ionic concentration in the synaptic cleft responsive to a glia.

27. The BMI of claim 26, wherein the atomic forcipes further comprises a transmitter configured to transmit a signal corresponding to the change in the ionic concentration in the synaptic cleft propagated from the glia.

28. The BMI of claim 27, wherein the atomic forcipes further comprises a receiver configured to receive a signal which initiates the change in the ionic concentration in the synaptic cleft propagated to the glia.

29. The BMI of claim 17, wherein the preselected nuclear magnetic isotope comprises an intercalated cation.

30. The BMI of claim 17, wherein the atomic forcipes has a length of between about 11 nanometers to about 20 nanometers.

31. The BMI of claim 29, wherein the intercalated cation comprises Fe+2 and the nuclear magnetic isotope comprises 57-Fe.

32. The BMI of claim 29 wherein the intercalated cation comprises Mn+2 and the nuclear magnetic isotope comprises 55-Mn.

33. The BMI of claim 29, wherein the intercalated cation comprises Co+2 and the nuclear magnetic isotope comprises 59-Co.

34. The BMI of claim 29, wherein the intercalated cation comprises Cu+2 and the nuclear magnetic isotope comprises 63-Cu and 65-Cu in a respective approximate atomic mass weight ratio of about 69:31.

35. A body-machine interface (BMI), comprising:
a biological structure comprising a biological intermediate, and
atomic forcipes coupled to, and disposed proximate to, the biological intermediate, wherein the atomic forcipes comprise:
at least one graphene sheet having a preselected concentration of deuterons as chemical adducts therein;
a piezoelectric clay sheet expressing a net negative surface charge and having paramagnetic properties, and abutted to the at least one graphene sheet; and
a gallery between the at least one graphene sheet and the clay sheet, the gallery having an adduct of a preselected nuclear magnetic isotope formed therein.

36. The BMI of claim 35, wherein the atomic forcipes is configured to be a sensor to detect a physical characteristic of the biological intermediate and a transmitter to wirelessly report a representation of the physical characteristic.

37. The BMI of claim 35, wherein the atomic forcipes is configured to be a receiver to wirelessly receive a representation of an action to be taken relative to the biological intermediate and an actuator to motivate the action in the biological structure.

38. The BMI of claim 36, wherein the atomic forcipes is configured to be a receiver to wirelessly receive a representation of an action to be taken relative to the biological intermediate and an actuator to implement the action in the biological intermediate.

39. The BMI of claim 38, wherein the biological structure comprises a first biological portion and a second biological portion with the biological intermediate therebetween, wherein the atomic forcipes obtains a physical characteristic representation of the biological intermediate and transmits the physical characteristic representation to a controller external to the biological structure.

40. The BMI of claim 39, wherein the biological structure is a knee joint, and the physical characteristic representation comprises one of joint configuration.

41. The BMI of claim 39, wherein the biological structure is a hip joint, and the physical characteristic representation comprises one of joint configuration.

42. The BMI of claim 39, wherein the biological structure is a shoulder joint, and the physical characteristic representation comprises one of joint configuration.

43. The BMI of claim 39, wherein the biological structure is a spinal joint, the first biological portion is a superior vertebra and the second biological portion is an inferior vertebra, relative to a longitudinal spinal axis, and the physical characteristic representation comprises one of joint configuration.

44. The BMI of claim 39, wherein the biological structure is one of an ankle or a wrist, and the physical characteristic representation comprises one of joint configuration.

45. The BMI of claim 38, wherein the biological structure comprises a heart, the biological intermediate comprises a selected portion of the myocardium, the physical characteristic is change in an electrical characteristic representative of at least a portion of a cardiac cycle sensed by the atomic forcipes, and the transmitter transmits the physical characteristic to an external controller.

46. The BMI of claim 45, wherein the atomic forcipes receives from the external controller an electrical characteristic representative of an electrical impulse to be imposed upon the myocardium intermediate, and actuates to impose the electrical impulse upon the selected portion of myocardium intermediate.

47. The BMI of claim 39, wherein the biological structure comprises a skeletal muscle, wherein the physical characteristic is a change in an electrical characteristic, and the change in an electrical characteristic causes the skeletal muscle to contract, relax, or alternatingly both.

48. The BMI of claim 47, wherein the skeletal muscle contracts one of isotonically, isometrically, or isokinetically.

49. The BMI of claim 39, wherein the intermediate is a skin wound with sutures and the physical characteristic representation of the biological intermediate is wound integrity, wound tension, wound infection, wound dehiscence, or wound healing.

50. The BMI of claim 49, wherein the biological structure is soft tissue or bone, wherein the physical characteristic is a change in an electrical characteristic, wherein the atomic forcipes receives from the external controller an electrical characteristic of an electrical waveform to be imposed on the wound and actuates to implement the electrical waveform in the wound to promote healing.

51. The BMI of claim 45, wherein the biological structure is soft tissue or bone, the biological intermediate is a cancer cell, the preselected nuclear magnetic isotope is a radioactive isotope, the atomic forcipes receives physical characteristic representation corresponding to release of the radioactive isotope from the gallery and actuates to release the radioactive isotope proximate to the cancer cell to kill the cancer cell.

52. The BMI of claim 45, wherein the biological structure is soft tissue or bone, the biological intermediate is a cancer cell, the preselected nuclear magnetic isotope is bound to an oncological pharmaceutical, the atomic forcipes receives physical characteristic representation corresponding to release of the oncological pharmaceutical and actuates to release the oncological pharmaceutical proximate to the cancer cell to kill the cancer cell.
